SEQUENCE LISTING (22 sequences)

SEQ ID NO: 1 - protein, 62 aa, Arabidopsis thaliana [sequence omitted]

SEQ ID NO: 2 - DNA, 189 bp, Arabidopsis thaliana
atgggaaaggtccacggttcattggctcgtgccggtaaggtgagaggtcagacacctaaagtggctaagcaggacaagaagaagaagcctcgtggtcgtgctcataagcgtctgcaacacaaccgccgtttcgtcaccgccgttgttggttttggcaagaagagaggaccaaactcatcagagaagtag

SEQ ID NO: 3 - DNA, 22 bp, Artificial Sequence
  Primer
ggaagttcatttattcggagag

SEQ ID NO: 4 - DNA, 22 bp, Artificial Sequence
  Primer
ggcaacaggattcaatcttaag

SEQ ID NO: 5 - DNA, 29 bp, Artificial Sequence
  Primer
ctggtcgacatgggaaaggtccacggttc

SEQ ID NO: 6 - DNA, 32 bp, Artificial Sequence
  Primer
tgatctagactacttctctgatgagtttggtc

SEQ ID NO: 7 - DNA, 29 bp, Artificial Sequence
  Primer
tgatctagacttctctgatgagtttggtc

SEQ ID NO: 8 - DNA, 22 bp, Artificial Sequence
  Primer
cacctaaagtggctaagcagga

SEQ ID NO: 9 - DNA, 19 bp, Artificial Sequence
  Primer
cttgccaaaaccaacaacg

SEQ ID NO: 10 - DNA, 22 bp, Artificial Sequence
  Primer
ttcataaccaatctcgatacac

SEQ ID NO: 11 - DNA, 21 bp, Artificial Sequence
  Primer
gacgcactttacttcgcaatc

SEQ ID NO: 12 - DNA, 22 bp, Artificial Sequence
  Primer
gatcaaaagcaatcactgcatc

SEQ ID NO: 13 - DNA, 20 bp, Artificial Sequence
  Primer
acggcccattaaagaggacc

SEQ ID NO: 14 - DNA, 20 bp, Artificial Sequence
  Primer
tcttggctgttggagacgac

SEQ ID NO: 15 - DNA, 24 bp, Artificial Sequence
  Primer
tctgagaagagttaaaagcgttgc

SEQ ID NO: 16 - DNA, 21 bp, Artificial Sequence
  Primer
acgaggattcttctgacgacg

SEQ ID NO: 17 - DNA, 23 bp, Artificial Sequence
  Primer
ccctaattctgagatttcgcacg

SEQ ID NO: 18 - DNA, 22 bp, Artificial Sequence
  Primer
ttctctgctttttgttcctccg

SEQ ID NO: 19 - DNA, 18 bp, Artificial Sequence
  Primer
cactttccagcagatgtg

SEQ ID NO: 20 - DNA, 20 bp, Artificial Sequence
  Primer
ttgtccgtcgggtaattcat

SEQ ID NO: 21 - DNA, 21 bp, Artificial Sequence
  Primer
tcgattgcttggtttgaaaga

SEQ ID NO: 22 - DNA, 25 bp, Artificial Sequence
  Primer
gcacttagcgtggactctgtttgat

(19) United States
(12) Patent Application Publication
(10) Pub. No.: US 2023/0220410 A1
(43) Pub. Date: Jul. 13, 2023

(54) PLANTS TOLERANT TO BROAD RANGE OF PATHOGENS

(71) Applicant: RAMOT AT TEL-AVIV UNIVERSITY LTD., Tel Aviv (IL)

(72) Inventors: Daniel CHAMOVITZ, Tel Aviv (IL); Alin FINKELSHTEIN, Tel Aviv (IL)

(21) Appl. No.: 18/001,626

(22) PCT Filed: Jun. 13, 2021
(86) PCT No.: PCT/IL2021/050710

§ 371 Date: Dec. 13, 2022

Related U.S. Application Data
(60) Provisional application No. 63/038,835, filed on Jun. 14, 2020.

(51) Int. Cl. C12N 15/82

(52) U.S. Cl. CPC C12N 15/8279

(57) ABSTRACT

The present invention relates to plants, particularly crop plants, having enhanced tolerance to biotic stress, particularly enhanced tolerance to stress caused by a wide range of plant pathogens. The tolerant plants of the invention show enhanced expression of Arabidopsis S30 protein or of homologs or orthologs thereof.

### FIELD OF THE INVENTION

The present invention relates to plants having enhanced expression of a ribosomal protein displaying increased tolerance to pathogen infection without having negative effects on the plant growth and yield.

### BACKGROUND OF THE INVENTION

Plants have evolved numerous chemicals to defend themselves against attack from herbivores and pathogens. One example is the glucosinolates (GLS) which are produced by members of the Brassicaceae family (Wittstock and Burow, 2010. The Arabidopsis Book 8, e0134-e0134). Upon tissue damage resulting from attack of chewing insects, necrotrophic pathogen infection, or other mechanical damage, GLS present in plant cells are hydrolyzed by thioglucosidase enzymes termed myrosinase (MYR), resulting in various indolic molecules (Andréasson E et al., 2001. Plant physiology 127, 1750-1763). The effects elicited by indole GLS hydrolysis products go beyond toxicity and range from mechanistic protection, such as increased callose deposition, strengthening of the cell wall and inhibition of cell penetration (Clay N K et al., 2009. Science 323, 95-101), to strategies such as modulation of the hypersensitive response (Zhao Y et al., 2015. The Plant Journal 81, 920-933).

Indole-3-carbinol (I3C) is one such GLS-breakdown product formed from the breakdown of indole-3-methylglucosinolate (I3M) (Wittstock and Burow, 2010, ibid). I3M is the predominant indole glucosinolate, and one of the most prominent glucosinolates detected in roots. The cleavage of I3M to I3C is mostly catalyzed by myrosinase (MYR) (Andreasson et al., 2001, ibid; Koroleva O A et al., 2010. The Plant Journal 64, 456-469); however, it can also be formed in a MYR-independent manner (Kim et al., 2008). I3C has a deleterious effect on herbivorous insects (Kim J H et al., 2008. The Plant Journal 54, 1015-1026; Li X et al., 2002. Insect Molecular Biology 11, 343-351) as well as considerable antimicrobial activity (Sung and Lee, 2007. Biological and Pharmaceutical Bulletin 30, 1865-1869).

An inventor of the present invention and co-workers have previously demonstrated that I3C serves as a signaling molecule in plants that modulates the auxin response by antagonizing auxin binding to the TIR1 receptor (Katz et al., 2015b. The Plant Journal 82, 547-555). I3C also affects the PIN1 and PIN2 auxin transporters (Katz et al., 2015a. Plant Signaling & Behavior 10, e1062200), and induces specific autophagy (Katz and Chamovitz, 2017. The Plant Journal 91, 779-787). Hence exogenous I3C at high concentrations inhibits Arabidopsis growth and development (Katz et al., 2015b, ibid).

The eukaryotic ribosome is a complex structure composed of four rRNAs and about 80 ribosomal proteins (r-proteins) (Barakat et al., 2001. Plant Physiology 127, 398), organized into two unequal subunits, the small subunit (40S) and large subunit (60S). Both of these subunits contain dozens of ribosomal proteins arranged on a scaffold composed of ribosomal RNA (rRNA). The small subunit monitors the complementarity between tRNA anticodons and mRNA, while the large subunit catalyzes peptide bond formation (Ben-Shem et al., 2011. Science 334, 1524-1529; Yusupova and Yusupov, 2017. Philosophical transactions of the Royal Society of London Series B, Biological sciences 372, 20160184). Arabidopsis r-protein genes and ribosome protein composition are very similar to those of other eukaryotes. In Arabidopsis thaliana, 249 r-protein genes were identified, encoding 32 and 48 putative small and large-subunit proteins, respectively. Due to the extensive segmental duplication of the Arabidopsis genome, all the r-protein genes have at least two, and often several, paralogues (Barakat et al., 2001. ibid).

Resistance of plants to pathogen infection, including pathogenic fungi and/or oomycetes, bacteria and insect has been the focus of extensive research, both for wide-range and pathogen-specific resistance mechanism of action. Large number of tolerance or resistance genes has been identified, as well as plant-defense response pathways. However, in cultivars with newly introduced resistance genes, protection from disease is often rapidly broken, due to pathogen adaptation and mutations leading to regained resistance. Also, wide areas of monocultures common in modern agriculture, increase the susceptibility of the crops to pathogens.

U.S. Pat. No. 6,573,361 discloses a protein isolated from Fusarium culmorum, termed FCWP1, which demonstrated significant antifungal activity against several fungal species. Mutations in proteolytic consensus sequences contained within FCWP1 improved the stability of its antifungal activity. A class of ribosomal proteins related to FCWP1 displayed similar values for pI and molecular weight compare to FCWP1. The disclosed antifungal proteins are useful in controlling fungal infections in plants, and transgenic plants may be produced that are more resistant to fungal infections relative to non-transgenic plants of the same species. Alternatively, the proteins may be applied to plants exogenously.

Publication of the inventors of the present invention, published after the priority date of the present invention, describes the identification and characterization of a novel Arabidopsis mutant tolerant to I3C. The mutant, designated ICT1, carries the AT2G19750 gene, which encodes an S30 ribosomal protein. Overexpression, but not knockout, of the S30 gene causes auxin-independent tolerance to I3C (Finkelshtein A et al., 2020. The Plant Journal 105, 668-677).

There is a constant need for new tolerance-conferring genes, particularly genes the expression of which does not have deleterious effects on the plant, specifically crop plant, growth and yield.

### SUMMARY OF THE INVENTION

The present invention answers the above-described needs, providing plants, particularly crop plants having enhanced expression and/or activity of an Arabidopsis S30 ribosomal protein and homologs thereof, displaying increased tolerance to a wide range of plant pathogens. Advantageously, the enhanced expression and/or activity of the S30 ribosomal protein does not negatively affect the plant growth and yield.

In the course of the research leading to the present invention, a search for Indole-3-carbinol (I3C)-tolerant plant by screening of Arabidopsis seed collection revealed an I3C-tolerant strain comprising AT2G19750 gene encoding an S30 ribosomal protein. Unexpectedly, plants having increased expression of the AT2G19750 gene exhibited increased tolerance to biotic stress, including tolerance to phytopathogenic fungi and bacteria. Without wishing to be bound by any specific theory or mechanism of action, the increased tolerance of the S30-ribosomal protein over-expressing plants may be further attributed to a modulated gene regulation exhibited by these plants. The modulated genes (which may be up-or down-regulated) are involved in intrinsic plant defense mechanisms against pathogens; the plants of the invention are thus primed to immediately combat the pathogen infection, therefore having enhances tolerance to a wide spectrum of pathogens.

According to one aspect, the present invention provides a plant or a part thereof comprising at least one cell modified to have enhanced expression and/or activity of a S30 protein compared to an unmodified cell, wherein the S30 protein comprises an amino acid sequence having at least 80% identity to the amino acid sequence set forth in SEQ ID NO:1, and wherein the plant has enhanced tolerance to at least one plant pathogen compared to a non-modified control plant.

According to certain embodiments, the S30 protein comprises an amino acid sequence at least 85%, at least 90%, at least 95%, or at least 98% identical to the amino acid sequence set forth in SEQ ID NO:1. According to certain embodiments, the S30 protein comprises the amino acid sequence set forth in SEQ ID NO:1.

According to certain embodiments, the S30 protein is encoded by a polynucleotide having a nucleic acid sequence at least 70% identical to the nucleic acid sequence set forth in SEQ ID NO:2. According to some embodiments, the S30 protein is encoded by a polynucleotide having a nucleic acid sequence at least 75%, at least 80%, at least 85%, at least 90%, at least 95%, or at least 98% identity to the nucleic acid sequence set forth in SEQ ID NO:2. According to some embodiments, the S30 protein is encoded by the polynucleotide having the nucleic acid sequence set forth in SEQ ID NO:2.

Enhancing the expression of the S30 protein in a plant cell may be achieved by various means, all of which are explicitly encompassed within the scope of the present invention.

According to certain embodiments, the at least one cell having enhanced expression and/or activity of the S30 protein comprises an exogenous polynucleotide encoding said S30 protein. The overexpressed polynucleotide can be endogenous or heterologous to the at least one plant cell.

According to certain embodiments, overexpression of an exogenous polynucleotide is affected by transforming at least one cell of the plant or part thereof with the heterologous polynucleotide. According to these embodiments, the plant is a transgenic plant comprising at least one cell transformed with an exogenous polynucleotide encoding a 30S protein having at least 80% identity to the amino acid sequence set forth in SEQ ID NO:1. According to certain embodiments, the transgenic plant comprises at least one cell comprising at least one polynucleotide having nucleic acid sequence at least 70% identical to SEQ ID NO:2.

According to certain embodiments, overexpression of a heterologous polynucleotide is affected by subjecting the at least one cell of the plant or part thereof to genome editing using artificially engineered nucleases as is known in the art.

Overexpression of an endogenous polynucleotide encoding the S30 protein can be affected at the genomic and/or the transcript level using a variety of methods that induce the transcription and/or translation of the polypeptide. According to certain embodiments, enhancing the expression of endogenous polynucleotide encoding the S30 protein is affected by mutating the endogenous polynucleotides, as long as the mutation results in enhanced expression and/or activity of said S30 protein. According to certain embodiments, enhancing the expression of endogenous polynucleotide encoding the S30 protein is affected by introducing a polynucleotide encoding a promoter or enhancer element in an appropriate position as to enhance transcription of said endogenous polynucleotide. According to yet further certain embodiments enhancing the expression of endogenous polynucleotide encoding the S30 protein is affected by subjecting the at least one cell of the plant or part thereof to genome editing using artificially engineered nucleases as is known in the art.

Since most genome-editing techniques can leave behind minimal traces of DNA alterations evident in a small number of nucleotides as compared to transgenic plants, crop plants created through gene editing for enhancing the expression of an endogenous or heterologous polynucleotide encoding the S30 protein could avoid the stringent regulation procedures commonly associated with genetically modified (GM) crop development, and are typically defined as non-transgenic crop plants.

According to certain embodiment, the expression and/or activity of the S30 protein or a polynucleotide encoding same within the at least one cell of the plant or part thereof is enhanced by at least 50%, i.e. the protein or polynucleotide level is at least 1.5-fold higher compared to its level in a control plant or compared to a predetermined threshold level. According to some embodiments, the level of the protein or polynucleotide expression is enhanced by at least 60%, 70%, 80%, 90%, 100%, 200%, 300% and more.

According to certain embodiments the plant pathogen is selected from phytopathogenic fungi, bacteria, insects, oomycetes, and nematodes. Each possibility represents a separate embodiment of the present invention.

According to certain embodiments, the fungal pathogens can be one or more fungi from a genus selected from the group consisting of Botrytis, Fusarium, Colletotrichum, Geotrichum, Aspergillus, Alternaria, Athelia, Botryosphaeria, Cryphonectria, Choanephora, Cercospora, Magnaporthe Monilinia, Mycosphaerella, Melampsora, Puccinia, Phakopsora, Rhizoctonia, Septoria, Uromyces, Ustilago and Verticillium. Each possibility represents a separate embodiment of the present invention.

According to certain exemplary embodiments, the fungal pathogen is Botrytis cinerea.

According to certain embodiments, the bacterial pathogen can be one or more bacteria of a genus selected from the group consisting of Pseudomonas, Ralstonia, Agrobacterium, Xanthomonas, Erwinia, Xylella, Dickeya, and Pectobacterium.

According to certain embodiments, the one or more bacteria are of a species selected from the group consisting of Pseudomonas syringae, Ralstonia solanacearum, Agrobacterium tumefaciens, Xanthomonas oryzae, Xanthomonas campestris pv, Xanthomonas axonopodis pv. Xanthomonas axonopodis pv., Erwinia amylovora, Xylella fastidiosa, Dickeya dadantii, Dickeya solani, and Pectobacterium carotovorum,

According to certain exemplary embodiments, the bacterial pathogen is of the genus Pseudomonas. According to additional exemplary embodiments, the bacterial pathogen is Pseudomonas syringae pv. tomato DC3000 (PST DC3000).

According to certain currently exemplary embodiments, the plant is a crop plant. According to some embodiments, the crop plant is selected from plant producing fruit; flower and ornamental plant; grain producing plant, including, but not limited to wheat, oats, barely, rye, rice, maize; legumes, including, but not limited to peanuts, peas, soybean, lentil; plant producing forage; plant producing fiber, including, but not limited to cotton, flax; a tree for wood industry; plant producing tuber or root crop; sugar beet; sugar cane; cannabis; plant producing oil, including, but not limited to canola, sunflower, sesame; and plants used for their leaves (lettuce, parsley, kale). Each possibility represents a separate embodiment of the present invention.

According to certain embodiments, the crop plant is of the family Solanaceae.

According to certain exemplary embodiments, the Solanaceae crop plant is selected from the group consisting of tomato (Solanum lycopersicum), eggplant (Solanum melongena) potato (Solanum tuberosum) and tobacco (Nicotiana tabacum). According to certain currently exemplary embodiments, the crop plant is a tobacco plant. According to certain additional currently exemplary embodiments, the crop plant is a tomato plant.

According to certain embodiments, the plant comprising at least one modified cell with enhanced expression and/or activity of a S30 protein comprises an amino acid sequence having at least 80% identity to the amino acid sequence set forth in SEQ ID NO:1 and having enhanced tolerance to at least one plant pathogen shows an equivalent growth rate and yield production compared to the growth rate and yield production of a corresponding non-modified plant grown under the same conditions.

The present invention further provides seeds, fruit, or any other part of the pathogen-tolerant plants of the invention, as well as tissue cultures derived thereof and plants regenerated therefrom.

According to certain embodiments, the present invention provides a seed of the pathogen-tolerant plant of the invention, wherein a plant grown from the seed comprises at least one cell modified to have enhanced expression and/or activity of a S30 protein, wherein the S30 protein comprises an amino acid sequence having at least 80% identity to the amino acid sequence set forth in SEQ ID NO:1.

According to another aspect, the present invention provides a method for conferring and/or enhancing tolerance of a plant or a part thereof to at least one plant pathogen, the method comprising enhancing the expression and/or activity of a S30 protein within at least one cell of the plant or part thereof, wherein the S30 protein comprises an amino acid sequence having at least 80% identity to the amino acid sequence set forth in SEQ ID NO:1, thereby conferring and/or enhancing tolerance of said plant or part thereof to the at least one pathogen compared to a control plant.

According to certain embodiments, enhancing the expression or activity of the 30S protein comprises enhancing the expression of a polynucleotide encoding said S30 protein within the at least one cell of the plant.

According to certain embodiments, enhancing the expression of the polynucleotide encoding the 30S protein comprises introducing into the at least one cell an exogenous polynucleotide encoding said S30 protein.

According to certain embodiments, introducing the exogenous polynucleotide comprises transforming the at least one cell with said polynucleotide or a construct comprising same, thereby producing a transgenic plant over-expressing the exogenous polynucleotide compared to a control non-transgenic plant.

According to certain embodiments, introducing the exogenous polynucleotide comprises subjecting the at least one cell to genome editing using artificially engineered nucleases.

The exogenous polynucleotide introduced into the at least one plant cell can be a polynucleotide endogenous to said plant cell or a polynucleotide heterologous to said plant cell.

According to yet further certain embodiments, enhancing the expression or activity of the 30S protein comprises up-regulating within the at least one cell of the plant or part thereof an endogenous polynucleotide encoding said S30 protein.

According to certain embodiments, up-regulating the expression of the endogenous polynucleotide comprises subjecting the at least one cell to genome editing using artificially engineered nucleases.

According to certain embodiments, up-regulating the expression of the endogenous polynucleotide comprises mutating the endogenous polynucleotide, wherein the mutation results in enhanced expression and/or activity of the encoded S30 protein.

According to yet further certain embodiments, up-regulating the expression of the endogenous polynucleotide comprises introducing to the at least one cell of the plant or part thereof a polynucleotide encoding a promoter or enhancer element in an appropriate position as to enhance transcription of said endogenous polynucleotide.

The polynucleotide encoding the S30 protein is as described hereinabove. According to certain embodiments, the polynucleotide comprises a nucleic acid sequence having the nucleic acid sequence set forth in SEQ ID NO:2.

According to certain embodiments, the control plant is a corresponding plant not manipulated to have enhanced expression and/or activity of the S30 protein in at least one of its cells. According to certain exemplary embodiments, the control plant is of the same species as the manipulated plant. According to further exemplary embodiments, the control plant has the same genetic background as the manipulated plant.

According to certain embodiments, the modified plant of the invention and the control plant have comparable growth rate and yield production.

According to certain embodiments, conferring and/or enhancing tolerance to at least one plant pathogen comprises preventing and/or treating a disease and/or deleterious symptoms caused by the at least one pathogen.

According to certain embodiments, the method comprises conferring and/or enhancing tolerance of a plurality of plants to at least one plant pathogen. According to these embodiments, the method further comprises selecting plants showing an enhanced tolerance to said at least one plant pathogen compared to a control plant or to a pre-determined tolerance score value.

According to certain embodiments, selecting plants showing enhanced tolerance to the at least one plant pathogen is performed by inoculating the plurality of plants with a respective plant pathogen and selecting phenotypically tolerant plants.

The control plants are as described hereinabove.

According to certain embodiments, the pre-determined tolerance score value is obtained by a method comprising the steps of inoculating a plurality of corresponding plants susceptible to the at least one plant pathogen; scoring the infection degree; and setting an average tolerance score value.

According to additional or alternative embodiments, selecting plants showing enhanced tolerance to the at least one plant pathogen is performed by detecting the presence of a S30 protein comprising an amino acid sequence having at least 80% identity to the amino acid sequence set forth in SEQ ID NO:1 or of a polynucleotide encoding same.

Any method as is known in the art for detecting the presence of a specific protein or a polynucleotide encoding same within a plant cell can be used according to the teachings of the present invention.

According to certain exemplary embodiments, detection is performed by identifying, in a genetic material obtained from the plant, the presence of a polynucleotide comprising a nucleic acid sequence having at least 70% identity to SEQ ID NO:2.

According to certain exemplary embodiments, detection is performed by identifying at least one sequence-specific probe that specifically hybridizes under stringent conditions to a nucleic acid sequence having at least 70% identity to the nucleic acid sequence set forth in SEQ ID NO:2.

According to certain exemplary alternative embodiments, detection is performed by identifying the presence of a polynucleotide amplified by a pair of primers comprising the nucleic acid sequence set forth in SEQ ID NO:5 and SEQ ID NO:6 or 7.

According to certain additional exemplary alternative embodiments, detection is performed by identifying the presence of a polynucleotide marker amplified by a pair of primers comprising the nucleic acid sequence set forth in SEQ ID NO:8 and SEQ ID NO:9. According to these embodiments, the amplified marker is indicative of the presence of an expressed S30 encoding polynucleotide (S30 encoding mRNA).

Also encompassed herein are plants produced by the methods of the invention, parts thereof and plants regenerated from same.

The plants, the at least one pathogen and the degree of enhanced tolerance is as described hereinabove.

According to yet further aspect, the present invention provides an isolated protein comprising an amino acid sequence having at least 80% identity to SEQ ID NO:1, wherein the isolated protein is effective in enhancing the tolerance of a plant to at least one plant pathogen.

According to certain embodiments, the isolated protein comprises an amino acid sequence having at least 85%, at least 90%, at least 95%, or at least 98% identity to the amino acid sequence set forth in SEQ ID NO:1. According to certain embodiments, the isolated protein comprises the amino acid sequence set forth in SEQ ID NO:1. According to certain embodiments, the isolated protein consists of the amino acid sequence set forth in SEQ ID NO:1.

According to further embodiments, the isolated protein is comprised within an agricultural composition suitable for applying the protein to a plant or a part thereof.

According to certain embodiments, the agricultural composition is a plant protection product effective in enhancing the tolerance of a plant to at least one plant pathogen. According to certain embodiments, the agricultural composition is effective in preventing or treating at least one plant disease or in reducing the symptoms caused by the at least one plant pathogen.

According to certain embodiments, the agricultural composition further comprises an agriculturally acceptable diluent(s) or carrier(s). According to certain embodiments, the agricultural composition further comprises at least one of a stabilizer, a tackifier, a preservative, a carrier, a surfactant or a combination thereof. Each possibility represents a separate embodiment of the present invention.

According to certain embodiments, the agricultural composition further comprises at least one additional active agent selected from the group consisting of a fertilizer, an additional pesticide, a plant growth regulator, a rodenticide, a nutrient and any combination thereof. Each possibility represents a separate embodiment of the present invention. According to certain embodiments, the at least one additional active agent is a synthetic agent.

The agricultural composition can be formulated in any form suitable for applying the composition to a plant or a part thereof as is known in the art. According to certain embodiments, the agricultural composition is formulated in a form selected from the group consisting of an emulsion, a colloid, a dust, a granule, a pellet, a powder, a spray, a pressurized form, a pressurizable form, and a solution. Each possibility represents a separate embodiment of the present invention.

According to some embodiments, the formulation is in a form selected from the group consisting of liquid, solid, semi-solid, gel or powder. Each possibility represents a separate embodiment of the present invention.

According to certain embodiments, the carrier is a plant seed. According to these embodiments, the present invention provides an agricultural composition comprising at least one plant seed and an isolated protein comprising an amino acid sequence having at least 80% identity to SEQ ID NO:1 in an amount effective in enhancing the tolerance of a plant grown from the seed to at least one plant pathogen.

According to certain embodiments, the agricultural composition is in a form of seed coating. According to these embodiments, the seed coating formulation further comprises at least one agent selected from the group consisting of a binding agent and a wetting agent. According to certain exemplary embodiments, the binding agent is carboxymethyl cellulose (CMC).

According to a further aspect, the present invention provides a method for enhancing and/or conferring tolerance of a plant or a part thereof to at least one plant pathogen, comprising contacting the plant or part thereof with an agricultural composition comprising a protein comprising an amino acid sequence having at least 90% identity to the amino acid sequence set forth in SEQ ID NO:1. According to certain embodiments, enhancing and/or conferring tolerance to at least one plant pathogen comprises preventing and/or treating a disease and/or deleterious symptoms caused by the at least one pathogen. According to certain embodiments, the plant is susceptible to the disease caused by the at least one pathogen. According to some embodiments, the method further comprises identifying symptoms of the disease within the plant or par thereof before administering the composition comprising the protein of the invention.

The agricultural composition, the plants and the at least one pathogen are described hereinabove.

Any method as is known in the art for administering the agricultural composition of the invention to a plant or to a part thereof can be used. According to some embodiments, the agricultural composition is formulated in a liquid form and the plant or part thereof may be contacted with the composition by a method selected from the group consisting of infiltration, immersion/dipping, incubation, spraying, and any combination thereof. Each possibility represents a separate embodiment of the present invention.

According to some embodiments, the plant part is a leaf and the agricultural composition is applied by spraying or dusting. According to some embodiments, the plant part is a root and the agricultural composition is applied by dipping or immersing.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 demonstrates that the I3CT1 plant (designated in the figure as ICT1) is tolerant to I3C. FIG. 1A: I3CT1 and wild type (WT, Col-0) grown on both MS and MS supplemented with 400 μM I3C. Scale bar-10 mm. FIG. 1B: Root lengths of I3CT1 and WT grown on either 0 or 400 μM I3C supplemented MS.* P=1.2E-08 (Student T-TEST). FIG. 1C: Developmental stages of WT versus I3CT1 grown for two weeks on MS (grey bars) or 400 μM I3C (black or dark-gray bars), and then transferred to soil pots.

FIG. 2 demonstrates the effect of AT2G19750 expression on tolerance to I3C. FIG. 2A: Expression levels of the S30 in AT2G19750 transgenic lines. FIG. 2B: Phenotype of transgenic lines on 400 μM I3C after 14 days. NC is a negative control plants transformed with empty plasmid (Scale bar—10 mm). FIG. 2C: The average root length on 400 μM I3C. * P<0.005 (Student T-TEST with equal variance). Col-0: wild type background of all transgenic lines. NC(8): transgenic lines with empty vector (negative control). ICT1 (1, 2, 28, 33): transgenic plants produced as exemplified hereinbelow. ICT1 (478, 8089): transgenic FOX overexpressing S30.

FIG. 3 shows the effect of I3C concentration on the growth rate of wild type vs. the transgenic plants of the invention. FIG. 3A: Growth rate of the WT versus I3CT1-2 and I3CT1-8098 (designated in the figure as ICT1-2 and ICT1-8098, respectively) on MS and on 400 μM I3C. The numbers on the right side indicate growth slops of each line. FIG. 3B: Inhibition curve in the presence of exogenic I3C. The insert table shows 50% and 90% inhibition in each plant strain.

FIG. 4 demonstrated that I3CT1 line is specifically tolerant to I3C and its derivatives. FIG. 4A: Plant defense molecules assayed: Breakdown products of indol-3-acetaldoxime (upper left panel), Breakdown products of I3C (lower panel), and benzoxazinoids (right panel). FIG. 4B: I3CT1 and WT were grown on 50% and 90% inhibition concentrations of the chemicals shown in μM. Data represent mean±SE.

FIG. 5 shows the results of qRT-PCR verification of RNA-Seq. To validate the levels of gene expression in RNA-Seq as reflected by the log 2 fold change values, 10 transcripts were selected and subjected to RT-qPCR. The fold change as seen in the qPCR reaction is compared to those found in the RNA-Seq experiment, for each gene.

FIG. 6 shows that genes involved in root development are down-regulated following treatment with I3C. 66 genes involved in root development were down-regulated in WT following treatment with 200 or 400 μM I3C, as part of an enriched GO grouping (GO:0022622 root development P 6.2E-5).

FIG. 7 demonstrates that genes involved in the glucosinolate pathway are downregulated following treatment with I3C. The enzymes and the transcription factors that are circled are down regulated in WT following treatment with I3C.

FIG. 8 shows the main pathways up- and down-regulated in WT and I3CT1 following treatment with I3C.

FIG. 9A shows cluster analysis of up- (left panel) and down- (right panel) regulated genes in WT following exposure to I3C at 200 or 400 μM, which also show modified expression in untreated transgenic line I3CT1² and I3CT1⁸⁰⁹⁸. The clusters of primed genes marked with the numbers are: 1—membrane and transport, 2—protein transport, 3—defense response, 4—secondary metabolites synthesis, 5—oxidative stress, 6—Glucosinolate biosynthesis, 7—transporters.

FIG. 9B shows the pathways affected in the primed responses.

FIG. 9C shows that I3CT1 plants (designated in the figure as ITC1) are resistant to 1, 5, and 10 mM H₂O₂ but not to salt and drought.

FIG. 9D shows infection of Col-0 and I3CT1 lines (designated in the figure as ITC1) with Botrytis cinerea. The lesions were measured 1 week after inoculation. Scale bar-1 cm.

FIG. 9E shows infection of Col-0 and I3CT1 lines (designated in the figure as ITC1) with Pseudomonas PST DC3000. There are 2.5 times more CFU in Col-0 then in I3CT1. n=9. The Asterisk (*) indicates the significant difference between Col-0 and I3CT1 mutants (one-tailed Student's t-test with equal variance, P<0.05 equal variance).

FIG. 10 demonstrates that plants overexpressing S30 retain I3C inhibition of IAA signaling. I3CT1², I3CT1⁸⁰⁹⁸ (FIG. 10A, designated as ICT1) and WT (FIG. 10B) plants were grown on three concentrations of I3C (50, 200, 500 μM), three concentrations of IAA (50, 200, 500 nM) and mixtures between the two. Root length was measured, and the mean root length of the transgenic lines I3CT1², I3CT1⁸⁰⁹⁸ was calculated (presented as “I3CT1”). Three-way ANOVA was performed after SQRT transformation to obtain a normal distribution. FIG. 10C: The model of inhibition.

### DETAILED DESCRIPTION OF THE INVENTION

The present invention discloses plants, specifically crop plants, which are modified to overexpress Arabidopsis ribosomal protein S30 or homologs/orthologs thereof, and have enhanced tolerance to plant pathogens. The present invention shows for the first time the involvement of the S30 protein in plant defense against pathogens. Without wishing to be bound by any specific theory or mechanism of action, protection against the plant pathogen may be attributed to the overexpression of the S30 protein as well as to an associated overexpression of other genes involved in plant defense mechanisms.

The present invention further provides compositions and methods for enhancing the tolerance and/or resistance of plants to phytopathogens.

### Definitions

The terms “comprise”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a plant pathogen” or “at least one plant pathogen” may include a plurality of plant pathogens, including mixtures thereof.

The term “about” as used herein refers to the numeric value it refers to ±10%.

When reference is made to particular sequence listings, such reference is to be understood to also encompass sequences that substantially correspond to its complementary sequence, as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

The term “plant” is used herein in its broadest sense. It includes, but is not limited to, any species of woody, herbaceous, perennial or annual plant. It also refers to a plurality of plant cells that are largely differentiated into a structure that is present at a stage of the plant development. According to certain currently exemplary embodiments, the plant of the invention is a crop plant. As used herein, the term “crop plant” refers to a plant with at least one part having commercial value. The term encompasses plants producing edible fruit (including vegetables), plants producing grains (as a food, feed and for oil production), plant producing flowers and ornamental plants, legumes, root crops, tuber crops, leafy crops and the like.

As used herein, the term “plant part” refers to a part of the plant, including single cells and cell tissues such as plant cells that are intact in plants, cell clumps and tissue cultures from which plants can be regenerated. Examples of plant parts include, but are not limited to, single cells and tissues from pollen, ovules, leaves, embryos, roots, root tips, anthers, flowers, fruits, stems shoots, and seeds; as well as pollen, ovules, leaves, embryos, roots, root tips, anthers, flowers, fruits, stems, shoots, scions, rootstocks, seeds, protoplasts, calli, and the like.

As used herein, the terms “tolerant” or “tolerance” with regard to plant pathogens refers to a plant having tolerance and/or resistance to infection by a plant pathogen or to the symptoms caused by a plant pathogen. For example, a plant tolerant and/or resistant to a plant pathogen can exhibit a lack of infection, or reduced symptoms of infection, when challenged with the pathogen. As another example, a plant tolerant and/or resistant to a plant pathogen can be infected by the pathogen and yet exhibit a reduced number or degree of symptoms of said infection. As yet another example, a plant tolerant and/or resistant to a pathogen can be infected by the pathogen and exhibit one or more symptoms of infection by the pathogen and yet exhibit a reduction in an effect of the infection or symptom thereof. For instance, a plant tolerant and/or resistant to a pathogen can be infected by the pathogen, and exhibit one or more symptoms selected from the group consisting of leaf wilt, leaf or vascular discoloration (e.g., yellowing), spike bleaching etc., and yet not exhibit a reduction in yield in comparison to the yield of a plant that is not affected by the pathogen grown under the same conditions.

Accordingly, “enhanced tolerance to a plant pathogen” or “conferred tolerance to a plant pathogen” refers to a phenotype in which a plant has greater health, growth, multiplication, fertility, vigor, strength (e.g., stem strength and resistance), yield, or less severe symptoms associated with infection of the pathogen than a plant that does not have enhanced tolerance to the pathogen. Where a plant is tested for tolerance, a control plant is used to assess the degree of the plant tolerance. According to certain embodiments of the present invention, the control plant is a plant not manipulated to have enhanced expression and/or activity of Arabidopsis S30 protein or an ortholog or homolog thereof. The control plant is typically, but not necessarily of the same species as the examined plant. According to some embodiments the control plant is of the same species and has the same genetic background as the examined plant. According to certain exemplary embodiments, the examined plant and the control plant are grown under the same conditions.

The enhanced or conferred tolerance can be manifested as a decrease of at least 5%, 6%, 7%, 8%, 9%, 10%, 12%, 15%, 17%, 20%, 25%, 30%, 35%, 40%, 50%, 60%, 70%, 80%, 90%, or 100% in the symptoms associated with the infection by the plant pathogen compared to the control plant.

The enhanced or conferred tolerance can additionally or alternatively be manifested as an increase of 0.1%, 0.2%, 0.3%, 0.5%, 0.75%, 1%, 1.5%, 2%, 3%, 4%, 5%, 6%, 7%, 8%, 9%, 10%, 12%, 15%, 17%, 20%, 25%, 30%, 35%, 40%, 50%, 60%, 70%, 80%, 90%, 100%, or more in health, growth, multiplication, fertility, vigor, strength, and/or yield, as compared to a control plant affected by the phytopathogen.

The terms “susceptible” and “susceptibility” as used herein refer to the inability of a plant to restrict the growth and development of a specified pathogen; a susceptible plant displays the detrimental symptoms linked to the pathogen infection

The terms “I3CT1”, “I3CT1 plant(s)”, “I3CT1 strain(s)” and “I3CT1 line(s)” are used herein interchangeably and refer to a plant/plants carrying AT2G19750 transgene or a homolog thereof overexpressing a 30S protein (having at least 80% identity to the amino acid sequence set forth in SEQ ID NO:1). Specific Arabidopsis transgenic lines are designated by number (e.g. I3CT1² or I3CT1(2), I3CT1³ or IsCT1(3) etc.). All terms of “I3CT1” in the instant specification and in the figures may also be referred to as “ICT1”

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene. The fragments may range in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein, the term “ortholog” refers to homologous genes in different species that evolved from a common ancestral gene. Accordingly, orthologs typically retain the same function during the course of evolution. In the context of the present invention, S30 ribosomal protein ortholog is a protein of a plant species other than Arabidopsis having the function of S30 ribosomal protein of enhancing and/or conferring to a plant tolerance towards at least one pathogen as disclosed in the present invention for the first time. According to certain embodiments, the S30 ribosomal protein ortholog comprises an amino acid sequence at least 80% identical to SEQ ID NO:1.

The terms “protein” and “polypeptide” are used interchangeably herein to designate a series of amino acid residues, connected to each other by peptide bonds between the alpha-amino and carboxy groups of adjacent residues. The terms “protein” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The term “isolated” refers to at least partially separated from the natural environment e.g., from a plant cell.

As used herein, “sequence identity” or “identity” in the context of two nucleic acid or amino acid sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage of sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of (Henikoff and Henikoff 1992).

Identity (e.g., percent homology) can be determined using any homology comparison software, including for example, the BlastN software of the National Center of Biotechnology Information (NCBI) such as by using default parameters.

Homology (e.g., percent homology, sequence identity+sequence similarity) can be determined using any homology comparison software computing a pairwise sequence alignment.

According to some embodiments of the invention, the identity or homology is a global identity/homology, i.e., an identity/homology over the entire amino acid or nucleic acid sequences of the invention and not over portions thereof.

According to some embodiments of the invention, the identity or homology is a partial identity/homology, i.e. an identity/homology over part of the amino acid or nucleic acid sequences of the invention. According to some embodiments, the partial sequence is of polynucleotides and polypeptides of embodiments of the present invention lacking a signal or transit peptide.

The term “exogenous” as used herein refers to a polynucleotide which is not naturally expressed within a plant cell (e.g., heterologous polynucleotide from a different plant species) or to an endogenous nucleic acid of which overexpression in the plant is desired. The exogenous polynucleotide may be introduced into the plant in a stable or transient manner, so as to produce a ribonucleic acid (RNA) molecule and/or a polypeptide molecule. The term “endogenous” as used herein refers to a polynucleotide or polypeptide which is naturally present and/or naturally expressed within a plant cell, e.g., the modified plant cell comprises an additional copy of the sequence. The term “heterologous” as used herein includes a sequence that was inserted to the plant cell and is not naturally found in the cell of this plant species.

The present invention discloses plants having enhanced tolerance to a wide range of plant pathogen, comprising at least one cell with enhanced expression of the AT2G19750 or homologs thereof encoding an S30 ribosomal protein, thereby enhancing the tolerance of the plant to a wide range of pathogens. The ability of the overexpressed S30 protein to enhance the tolerance of a plant to pathogen infection has been unexpectedly discovered during a course of a research for understanding how indole-3-carbinol (I3C), previously identified as a signaling molecule which modulates auxin signaling and playing a key role in the defense against plant attackers (Katz and Chamovitz, 2017. ibid; Katz et al., 2015a. ibid; Katz et al., 2015b. ibid), interfaces with plant metabolism and growth.

A genetic approach to isolate strains tolerant to I3C treatment was taken. Following the screening of 20,000 seeds from the full-length cDNA Over-eXpression (FOX) library (Takanari et al., 2006. The Plant Journal 48, 974-985), one strain with stable tolerance to I3C was isolated and designated I3CT1 or ICT1. I3CT1 carries a transgene over-expressing AT2G19750, which encodes the S30 subunit of the 40S ribosome.

In lieu of a functional description for S30, a transcript profiling was employed in an attempt to understand the influence of S30 overexpression on the plant, as well as to describe the transcriptional effects of I3C exposure on the WT. The transcript levels of several hundred genes changed following a two-hour exposure to I3C. At a global level, exposure to I3C influences the expression of a number of genes involved in various phytohormone pathways, down-regulating genes related to mostly aliphatic, but also indolic, glucosinolates, auxin and root development. However, as exemplified herein for the first time, expression of genes involved in various defense responses have been induced. An increase in the concentration of I3C treatment did not lead to increases in the magnitude of changes in expression, nor in the expression of novel gene groups. Rather, the increased concentration of I3C led to modified expression of additional genes in the pathways affected by the lower concentration. Thus, exposure to high doses of I3C apparently leads to increased growth arrest due to the modified expression of numerous genes in the same pathways regulated by low I3C concentrations.

While it was predicted that the tolerance to I3C may be reflected in differences in the genes the expression of which has been modified following a brief exposure to I3C, in practice, many of these same genes were also found to have modified expression in I3CT1 following a two-hour exposure to I3C. However, it was noticed that many of these same genes show modified expression in I3CT1 plants, relative to the WT, under normal growth conditions. That is, genes having modified expression in WT upon exposure to I3C, show an equivalent modified expression in I3CT1 plants under normal conditions. Thus, overexpression of S30 apparently leads to a change in gene expression, such that the plant is primed to respond to I3C. Unexpectedly, the primed genes include those involved in plant responses to oxidative and biotic stress, as confirmed by tolerance of I3CT1 plants to oxidative and biotic challenge. Accordingly, the present invention discloses that overexpressing in a plant a subunit of the ribosome, particularly the S30 protein not only lead to tolerance to I3C, but also to several environmental challenges. Specifically, priming genes involved in plant defense response towards pathogens, confers to or enhances the tolerance of the plant overexpressing the 30S protein of the invention towards a wide range of plant pathogens.

The terms “plant pathogen” and “pathogen” in singular or plural are used herein interchangeably and refers to any kind of pathogen, including viruses, bacteria, nematodes, fungi, oomycetes and insects the interaction of which with a plant results in a plant disease and/or deleterious symptoms.

The roles of individual component of the 40S ribosomal subunit are not well characterized (Wool, I G 1996. Trends Biochem Sci 21, 164-165). Studies have indicated that individual ribosomal proteins have extra-ribosomal functions in addition to those in the translation machinery (Lu H et al., 2015. Microbiol Res 177, 28-33; Nadano D et al., 2000. Jpn J Cancer Res 91, 802-810; Warner and McIntosh, 2009. Mol Cell 34, 3-11). Interestingly, higher expression of S30 protein is human correlated with breast cancer susceptibility (Zhang Y et al., 2018. Cancer Res 78, 1579-1591). Moreover, S30 shows differential expression in different cancer lines. However, this doesn't affect ribosomal structure and function, rather, this difference appears to be related to the extra-ribosomal function of S30 (Nadano et al., 2000, ibid). Without wishing to be bound by any specific theory or mechanism of action, a possible extra-ribosomal function of S30 could be as an antimicrobial peptide (AMP). AMPs are found in all multicellular organisms and Ubiquicidin, an AMP isolated from murine macrophages (Hiemstra P S et al., 1999. Journal of Leukocyte Biology 66, 423-428), is identical to mouse and human S30 (Wiesner and Vilcinskas, 2010. Virulence 1, 440-464).

Thus, according to one aspect, the present invention provides a plant or a part thereof comprising at least one cell modified to have enhanced expression and/or activity of a S30 protein compared to an unmodified cell, wherein the S30 protein comprises an amino acid sequence having at least 80% identity to the amino acid sequence set forth in SEQ ID NO:1, and wherein the plant has enhanced tolerance to at least one plant pathogen compared to a non-modified control plant.

As used herein, the expression and/or activity of the S30 protein of the invention is “enhanced” or “upregulated” if the level of the protein or its measured activity, or the polynucleotide encoding same is enhanced by at least 50%, i.e. the level of the polynucleotide, polypeptide, or the protein activity is at least 1.5-fold higher compared to its level in a control plant or compared to a predetermined threshold level. According to some embodiments, the level of the polynucleotide or polypeptide expression, or the protein activity is enhanced by at least 60%, 70%, 80%, 90%, 100%, 200%, 300% and more.

According to certain embodiments, the S30 protein comprises an amino acid sequence least at least at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more homologous to, or identical to the amino acid sequence forth in SEQ ID NO:1. According to certain embodiments, the S30 protein consists of the amino acid sequence set forth in SEQ ID NO:1.

According to certain embodiments, the S30 protein is encoded by a polynucleotide have a nucleic acid sequence at least 70%, at least about 71%, at least about 72%, at least about 73%, at least about 74%, at least about 75%, at least about 76%, at least about 77%, at least about 78%, at least about 79%, at least about 80%, at least 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more homologous to, or identical to the amino acid sequence forth in SEQ ID NO:2. According to certain embodiments, the S30 protein is encoded by a polynucleotide consisting of the nucleic acid sequence set forth in SEQ ID NO:2.

According to another aspect, the present invention provides a method for producing a plant with enhanced tolerance to at least one pathogen, the method comprising enhancing the expression and/or activity of a S30 protein within at least one cell of the plant or part thereof, wherein the S30 protein comprises an amino acid sequence having at least 80% identity to the amino acid sequence set forth in SEQ ID NO:1, thereby producing a plant with enhanced tolerance to the at least one pathogen compared to a control plant.

According to certain embodiments, the method comprises conferring and/or enhancing tolerance of a plurality of plants to at least one plant pathogen. According to these embodiments, the method further comprises selecting plants showing an enhanced tolerance to said at least one plant pathogen compared to a control plant or to a pre-determined tolerance score value.

Enhancing the expression and/or activity of the S30 protein can be perfected by any method as is known in the art, including transforming the at least one plant cell with exogenous polynucleotide encoding the S30 protein; introducing into the at least one plant cell an exogenous polynucleotide encoding the S30 protein by genome editing; modulating the expression of an endogenous polynucleotide encoding the S30 protein by subjecting the cell to genome editing; mutating the endogenous polynucleotide to have higher expression or to encode a protein with higher activity; transform the at least one cell with a polynucleotide encoding regulating element enhancing the expression of endogenous or exogenous polynucleotide encoding the S30 protein; modulating post-translational modifications to enhance the activity of the translated protein; and the like.

Methods of transforming a plant cell with a polynucleotide encoding a protein of interest are known in the art.

As used herein the term “transformation” or “transforming” describes a process by which a foreign nucleic acid sequence, such as a vector, enters and changes a recipient cell into a transformed, genetically modified or transgenic cell. Transformation may be stable, wherein the nucleic acid sequence is integrated into the plant genome and as such represents a stable and inherited trait, or transient, wherein the nucleic acid sequence is expressed by the cell transformed but is not integrated into the genome, and as such represents a transient trait. According to typical embodiments the nucleic acid sequences of the present invention are stably transformed into a plant cell.

There are various methods of introducing foreign nucleic acid sequences into both monocotyledonous and dicotyledonous plants (for example, Potrykus I. 1991. Annu Rev Plant Physiol Plant Mol Biol 42:205-225; Shimamoto K. et al., 1989. Nature 338:274-276).

The principal methods of the stable integration of exogenous DNA into plant genomic DNA includes two main approaches:

Agrobacterium-mediated gene transfer: The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation (Horsch et al., 1988. Plant Molecular Biology Manual A5, 1-9, Kluwer Academic Publishers, Dordrecht). A supplementary approach employs the Agrobacterium delivery system in combination with vacuum infiltration. The Agrobacterium system is especially viable in the creation of transgenic dicotyledonous plants.

Direct nucleic acid transfer: There are various methods of direct nucleic acid transfer into plant cells. In electroporation, protoplasts are briefly exposed to a strong electric field, opening up mini-pores to allow DNA to enter. In microinjection, the nucleic acid is mechanically injected directly into the cells using micropipettes. In microparticle bombardment, the nucleic acid is adsorbed on microprojectiles such as magnesium sulfate crystals or tungsten particles, and the microprojectiles are physically accelerated into cells or plant tissues. Another method for introducing nucleic acids to plants is via the sonication of target cells. Alternatively, liposome or spheroplast fusion has been used to introduce expression vectors into plants.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced from the seedlings to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Plants of the invention producing by transforming a polynucleotide encoding the 30S protein of the invention are defined as transgenic plants.

Enhancing the expression and/or activity of the S30 protein of the invention within a plant cell can be also achieved by means of genome editing. Genome editing is a reverse genetics method which uses artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

The CRISPR/Cas system for genome editing contains two distinct components: a gRNA (guide RNA) and an endonuclease e.g., Cas9.

The gRNA is typically a 20-nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Comparable with other genome editing nucleases, Zinc-finger nucleases (ZFNs) and transcription activator-like effector nucleases (TALENs), the double-stranded brakes produced by CRISPR/Cas can undergo homologous recombination or nonhomologous end-joining (NHEJ).

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present bi-allelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or homology directed repair (HDR) depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are number of publicly available tools to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids.

Genome editing is a powerful tool to impact target traits by modifications of the target plant genome sequence. Such modifications can result in new or modified alleles or regulatory elements.

Over-expression of a polypeptide by genome editing can be achieved by: (i) replacing an endogenous sequence encoding the polypeptide of interest or a regulatory sequence under which it is placed, and/or (ii) inserting a new gene encoding the polypeptide of interest in a targeted region of the genome, and/or (iii) introducing point mutations which result in up-regulation of the gene encoding the polypeptide of interest (e.g., by altering the regulatory sequences such as promoter, enhancers, 5′-UTR and/or 3′-UTR, or mutations in the coding sequence).

The minimal traces of DNA alterations evident in a small number of nucleotides obtained using gene editing techniques as compared to transgenic plants, crop plants created through gene editing for enhancing the expression of an endogenous or heterologous polynucleotide encoding the S30 protein may be defined as non-transgenic crop plants.

According to yet another aspect, the present invention provides a method for producing a population of plants each having enhanced tolerance to at least one pathogen, comprising the steps of:
a. enhancing the expression and/or activity of a S30 protein comprising an amino acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more identity, or identical to the amino acid sequence set forth in SEQ ID NO:1 within at least one cell of each plant of the plant population to produce a plurality of genetically engineered plant population;
b. inoculating each plant of the genetically engineered plant population with the at least one pathogen; and
c. selecting plants showing an enhanced tolerance to said at least one pathogen compared to a control plant or to a pre-determined tolerance score value;

thereby producing a population of genetically engineered plants having enhanced resistance to said at least one pathogen.

Enhancing the expression and/or activity of the 30S protein is as described hereinabove.

According to certain embodiments, selecting plants showing enhanced tolerance to the at least one plant pathogen is performed by inoculating the plurality of plants with a respective plant pathogen and selecting phenotypically tolerant plants.

The control plants are as described hereinabove.

According to certain embodiments, the pre-determined tolerance score value is obtained by a method comprising the steps of inoculating a plurality of corresponding plants susceptible to the at least one plant pathogen; scoring the infection degree; and setting an average tolerance score value.

According to additional or alternative embodiments, selecting plants showing enhanced is performed by detecting the presence of S30 protein comprising an amino acid sequence having at least 80% identity to the amino acid sequence set forth in SEQ ID NO:1 or of a polynucleotide encoding same.

Once expressed within the plant cell or the entire plant, the level of the 30S protein of the invention can be determined by methods well known in the art such as, activity assays, Western blots using antibodies capable of specifically binding the polypeptide, Enzyme-Linked Immuno Sorbent Assay (ELISA), radio-immuno-assays (RIA), immunohistochemistry, immunocytochemistry, immunofluorescence and the like.

Any method as is known in the art for detecting the presence of the polynucleotide encoding the 30S protein of the invention within a plant cell can be used according to the teachings of the present invention.

According to certain exemplary embodiments, detection is performed by identifying, in a genetic material obtained from the plant, the presence of a polynucleotide comprising a nucleic acid sequence having at least about 70%, at least about 71%, at least about 72%, at least about 73%, at least about 74%, at least about 75%, at least about 76%, at least about 77%, at least about 78%, at least about 79%, at least about 80%, at least 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or more identity, or identical, to SEQ ID NO:2.

According to certain exemplary embodiments, detection is performed by identifying at least one sequence-specific probe that specifically hybridizes under stringent conditions to a nucleic acid sequence having at least 70% identity to the nucleic acid sequence set forth in SEQ ID NO:2.

According to certain exemplary alternative embodiments, detection is performed by identifying the presence of a polynucleotide amplified by a pair of primer comprising the nucleic acid sequence set forth in SEQ ID NO:5 and SEQ ID NO:6 or 7. According to certain additional exemplary alternative embodiments, detection is performed by identifying the presence of an RNA polynucleotide encoding the S30 protein amplified by a pair of primer comprising the nucleic acid sequence set forth in SEQ ID NO:8 and SEQ ID NO:9.

According to yet further aspect, the present invention provides an isolated protein comprising an amino acid sequence having at about least 80% identity to SEQ ID NO:1, wherein the isolated protein is effective in enhancing the tolerance of a plant to at least one plant pathogen.

According to certain embodiments, the isolated protein comprises an amino acid sequence having at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98% or at least about 99% identity to the amino acid sequence set forth in SEQ ID NO: 1. According to certain embodiments, the isolated protein comprises the amino acid sequence set forth in SEQ ID NO:1. According to certain embodiments, the isolated protein consists of the amino acid sequence set forth in SEQ ID NO:1.

According to further embodiments, the isolated protein is comprised within an agricultural composition suitable for applying the protein to a plant or a part thereof.

According to certain embodiments, the agricultural composition is a plant protection product effective in enhancing the tolerance of a plant to at least one plant pathogen. According to certain embodiments, the agricultural composition is effective in preventing or treating at least one plant disease or in reducing the symptoms caused by the at least one plant pathogen.

According to certain embodiments, the agricultural composition further comprises an agriculturally acceptable diluent(s) or carrier(s). According to certain embodiments, the agricultural composition further comprises at least one of a stabilizer, a tackifier, a preservative, a carrier, a surfactant or a combination thereof. Each possibility represents a separate embodiment of the present invention.

According to certain embodiments, the agricultural composition further comprises at least one additional active agent selected from the group consisting of a fertilizer, an additional pesticide, a plant growth regulator, a rodenticide, a nutrient and any combination thereof. Each possibility represents a separate embodiment of the present invention. According to certain embodiments, the at least one additional active agent is a synthetic agent.

The agricultural composition can be formulated in any form suitable for applying the composition to a plant or a part thereof as is known in the art. According to certain embodiments, the agricultural composition is formulated in a form selected from the group consisting of an emulsion, a colloid, a dust, a granule, a pellet, a powder, a spray, a pressurized form, a pressurizable form, and a solution. Each possibility represents a separate embodiment of the present invention.

According to some embodiments, the formulation is in a form selected from the group consisting of liquid, solid, semi-solid, gel or powder. Each possibility represents a separate embodiment of the present invention.

According to certain embodiments, the carrier is a plant seed. According to these embodiments, the present invention provides an agricultural composition comprising at least one plant seed and an isolated protein comprising an amino acid sequence having at least 80% identity to SEQ ID NO:1 in an amount effective in enhancing the tolerance of a plant grown from the seed to at least one plant pathogen.

According to certain embodiments, the agricultural composition is in a form of seed coating. According to these embodiments, the seed coating formulation further comprises at least one agent selected from the group consisting of a binding agent and a wetting agent. According to certain exemplary embodiments, the binding agent is carboxymethyl cellulose (CMC).

According to a further aspect, the present invention provides a method for enhancing and/or conferring tolerance of a plant or a part thereof to at least one plant pathogen, comprising contacting the plant or part thereof with an agricultural composition comprising a protein comprising an amino acid sequence having at least 90% identity to the amino acid sequence set forth in SEQ ID NO: 1. According to certain embodiments, enhancing and/or conferring tolerance to at least one plant pathogen comprises preventing and/or treating a disease and/or deleterious symptoms caused by the at least one pathogen. According to certain embodiments, the plant is susceptible to the disease caused by the at least one pathogen. According to some embodiments, the method further comprises identifying symptoms of the disease within the plant or par thereof before administering the composition comprising the protein of the invention.

The agricultural composition, the plants and the at least one pathogen are described hereinabove.

Any method as is known in the art for administering the agricultural composition of the invention to a plant or to a part thereof can be used. According to some embodiments, the agricultural composition is formulated in a liquid form and the plant or part thereof may be contacted with the composition by a method selected from the group consisting of infiltration, immersion/dipping, incubation, spraying, and any combination thereof. Each possibility represents a separate embodiment of the present invention.

According to some embodiments, the plant part is a leaf and the agricultural composition is applied by spraying or dusting. According to some embodiments, the plant part is a root and the agricultural composition is applied by dipping or immersing.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

Materials and Methods

Plant Material and Growth Assays

The Arabidopsis strains used were all in the Columbia-0 (Col-0) background. The gain of function lines psx00478 and psx08098 containing pBIG2113SF binary vector, expressing a single cDNA under 35S promoter, were ordered from FOX library (Takanari I. et al., 2006. ibid). Transgenic lines were generated using pBINPLUS (van Engelen F A et al., 1995. Transgenic Research 4, 288-290), in which AT2G19750 gene was cloned downstream of the 35S promoter, and transformed to WT Col-0 via Agrobacterium cv3101 mediated transformation. The presence of the transgene was confirmed by PCR with specific primers for S30. Seeds were cultivated in Petri plates using medium containing 0.8% agar, half-strength Murashige and Skoog salts (MS), and 1% sucrose, pH 5.7. The Petri plates were placed in chambers at 22° C. under light/dark conditions of 16 h white light at 75 mol m² sec⁻¹ and 8 h darkness, at 55% relative humidity. For root phenotype experiments, plates were placed vertically in the chambers.

Indole-3-carbinol (I3C), was dissolved in dimethylsulfoxide to produce 1 M solution, and stored in the dark at −20° C.

### Screening

The FOX library was obtained from RIKEN Japan. Seeds were sown on 500 μM I3C. 20,000 different FOX lines were screened in pools of 50. Seedlings with longer roots than wild type (WT) were isolated and assayed two more times for root length on 500 μM I3C. To exclude the possibility of long root mutants, putative I3C-tolerant lines were sown on both 500 μM I3C and MS plates. Only lines that presented longer roots in presence of I3C but not on MS were selected for further analysis.

### Cloning

S30 cDNA was cloned in the sense orientation into the binary vector pBINPLUS (van Engelen et al., 1995, ibid) between the 35S-Ω promoter containing the translation enhancer signal and the Nos terminator, generating Pro35S:S30 construct. The original cloning vector pBINPLUS served as a control where applicable. Primers S30 forward and revers 1/2 were used to clone S30. The constructs were electroporated into Agrobacterium tumefaciens GV3101. Col-0 was used for transformation with Agrobacterium.

The primers used are listed in Table 1 hereinbelow. Table 1: Primers used for cloning

| SEQ ID | | | |
|---|---|---|---|
| No. | Primer name | Sequence | Description |
| 3 | F1 (FOX for) | 5′GGAAGTTCATTTATTCGGAGAG3′ | FOX specific |
| | | | primers |
| 4 | R1 (FOX rev) | 5′GGCAACAGGATTCAATCTTAAG3′ | |
| 5 | S30 for | 5′CTGGTCGACATGGGAAAGGTCCAC | S30 CDS |
| | | GGTTC3′ | primers |
| 6 | S30 rev 1 | 5′TGATCTAGACTACTTCTCTGATGA | With STOP |
| | | GTTTGGTC3′ | codon |
| 7 | S30 rev 2 | 5′TGATCTAGACTTCTCTGATGAGTT | Without |
| | | TGGTC3′ | STOP codon |
| 8 | 30S qPCR_LP | 5′CACCTAAAGTGGCTAAGCAGGA3′ | AT2G19750 |
| | | | qPCR exon |
| | | | 2, 3 |
| 9 | 30S qPCR_RP | 5′CTTGCCAAAACCAACAACG3′ | |
| 10 | Sail LB | 5′TTCATAACCAATCTCGATACAC3′ | Sail general |
| | | | primer |
| 11 | 30S_178_LP | 5′GACGCACTTTACTTCGCAATC3′ | Sail specific |
| | | | primers |
| 12 | 30S_178_RP | 5′GATCAAAAGCAATCACTGCATC3′ | |
| 13 | qPCR 30s | 5′ACGGCCCATTAAAGAGGACC3′ | AT2G19750 |
| | cDNA for | | qPCR 5′UTR |
| 14 | qPCR 30s | 5′TCTTGGCTGTTGGAGACGAC3′ | |
| | cDNA rev | | |
| 15 | AT4_qPCR_F | 5′TCTGAGAAGAGTTAAAAGCGTTGC | AT4G29390 |
| | | 3′ | qPCR |
| 16 | AT4_qPCR_R | 5′ACGAGGATTCTTCTGACGACG3′ | |
| 17 | At5_qPCR_F | 5′CCCTAATTCTGAGATTTCGCACG | AT5G56670 |
| | | 3′ | qPCR |
| 18 | AT5_qPCR_R | 5′TTCTCTGCTTTTTGTTCCTCCG3′ | |
| 19 | Actin_for_RT | 5′CACTTTCCAGCAGATGTG3′ | qPCR |
| | | | negative |
| | | | control |
| 20 | Actin_rev_RT | 5′TTGTCCGTCGGGTAATTCAT3′ | 18 |
| 21 | Clatrin_for | 5′TCGATTGCTTGGTTTGAAAGA3′ | qPCR |
| | RT | | negative |
| | | | control |
| 22 | Clatrin_rev | 5′GCACTTAGCGTGGACTCTGTTTGA | |
| | RT | T3′ | |

### Calculation of Root Growth Inhibition

To calculate the percentage of growth inhibition, seeds were germinated on MS containing the following concentrations of I3C: 0, 50, 100, 200, 300, 400, 500 and 750 μM. The root length of the seedlings was measured at days 3, 4, 5, 7 and 10. To calculate the percentage of growth inhibition, the slope of the regression lines for each concentration was used in the next equation:

$$( 1 - \frac{Slope of linear regression for specific I 3 C concentration}{Slope of linear regression on 0 μ M I 3 C ( MS )} )$$

### Statistics

The variance between the groups that were statistically compared was similar. The samples were analyzed using TTEST. p<0.05 was considered as significant.

### Phenotyping

Seeds of Col-0, and transgenic plants of the invention overexpressing the 30S protein (“gain of function” plants) were sown and grown for two weeks on MS agar versus 400 μM I3C plates. Following initial exposure to I3C the seedlings were transferred to soil pots. The plants were measured weekly for 100 days.

### RNA-Seq

Three lines were used for Next Generation Sequencing (NGS): Col-0 (control), and the 30S overexpressing transgenic lines I3CT1² and I3CT1⁸⁰⁹⁸. The seedlings were grown hydroponically for 10 days. Pooling of 20 seedlings were used for each sample, three biological samples were collected for each treatment. In all treatments, solutions of DMSO, 200 & 400 μM of I3C were prepared in MS medium, added to the liquid medium of hydroponic growth and incubated for 2 hours. All samples were collected at 16:00 (to eliminate the circadian clock effect). Table 2 summarizes the treatment groups.

TABLE 2
NGS treatment groups

| | treatment | | |
|---|---|---|---|
| Sample | NC (DMSO) | I3C 200 μM | I3C 400 μM |
| Col-0 | 3 | 3 | 3 |
| I3CT1² | 3 | 3 | 3 |
| I3CT1⁸⁰⁹⁸ | 3 | 3 | |

NC: negative control - wilt type Col-0 plant transformed with empty vector

Roots were cut and the RNA was extracted with Trizol following zymo RNA mini prep quick (R2072). 1500 μg RNA were used for libraries preparation. 24 RNA libraries were generated using ‘Illumina TruSeq RNA Library Preparation Kit v2’ according to manufacturer's protocol, and sequenced on Illumina HiSeq2500, 50 single-end run. An average of 20 million reads were sequenced per sample. The reads were mapped to the TAIR10 genome (ftp://ftp.arabidopsis.org/home/tair/Genes/TAIR10_genome_release/TAIR10_chromoso me_files/) using Tophat2 version 2.1.0 (ncbi.nlm.nih.gov/pubmed/19289445?dopt=Abstract), with up to 3 mismatches allowed per read. The minimum and maximum intron sizes were set to 11 and 11,000, respectively, and an annotation file was provided to the mapper. Only uniquely mapped reads were counted to exons, using ‘HTSeq-count’ package version 0.6.1 with ‘union’ mode (ncbi.nlm.nih.gov/pubmed/25260700?dopt=Abstract). Normalization and differential expression analyses were conducted using DESeq2 R package version 1.18.1 (ncbi.nlm.nih.gov/pubmed/25516281?dopt=Abstract).

qRT-PCR

The Quantabio Kit (95047-100) was used to synthesize the cDNA from the total RNA. Each RT-qPCR reaction was set up in a 15-μl volume containing 0.6 μl of cDNA, 0.4 μl of gene-specific primers, 7.5 μl of SYBR (Quantabio 65072-012) and 6.1 μl of sterile distilled water. Actin and PPR (AT1G49240 & AT1G62930) were used as the reference genes. Each reaction included three biological replicates, which were analyzed via StepOne software, using the 2\hat{ }ΔΔCt method for relative abundance calculation. The experiment was repeated 3 times.

Infection with Botrytis cinerea

B. cinerea spores were diluted to 5×10⁵ spores/mL in 0.53% potato dextrose broth. Droplets (7 μL) of 0.53 potato dextrose broth with 10⁵ B. cinerea spores were deposited on leaf surfaces of 4-week-old Arabidopsis plants (three leaves per plant). After incubation of the inoculated plants at high humidity for 7 days, the size of the disease lesion was measured. At least 15 lesion diameters were evaluated for each independent treatment (five plants). The lesion area was then measured with ImageJ.

Infection with Pseudomonas

Five-week-old Arabidopsis plants (Col-0, I3CT1² and I3CT1⁸⁰⁹⁸) were inoculated with Pseudomonas PST DC3000 WT (Pst) by infiltration with a suspension (1×10⁵ CFU/mL) of Pst in a 10 mM MgCl₂ solution using a needleless syringe. Three 1-cm-diameter leaf discs were sampled at 0, 3 and 6 days post-inoculation (dpi) from five inoculated plants and ground in 1 mL of 10 mM MgCl₂. Samples were then 10-fold serially diluted and plated on LB plates supplemented with 25 mM rifampicin. The colony counts were recorded 2 days after incubation at 28° C.

### Example 1: Isolation of an Arabidopsis Line Tolerant to I3C

Indole-3-carbinol (I3C) influences plant growth and development by manipulating auxin signaling. Phenotypically, this is easily seen by inhibition of root elongation following exogenous I3C treatment (Katz E. et al., 2015b. ibid). To explore how exposure to I3C affects plant growth and development, screening for Arabidopsis strains that are tolerant to high concentrations of exogenous I3C was performed. For this purpose, the Full-length cDNA Over-eXpression (FOX) library (Takanari et al., 2006, ibid) was screened on 400 μM I3C, which inhibits 95% of root elongation (Katz et al., 2015, ibid) for strains with relatively longer roots.

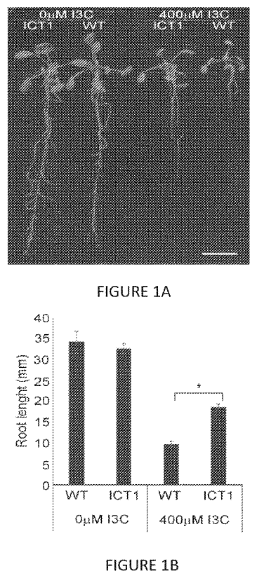

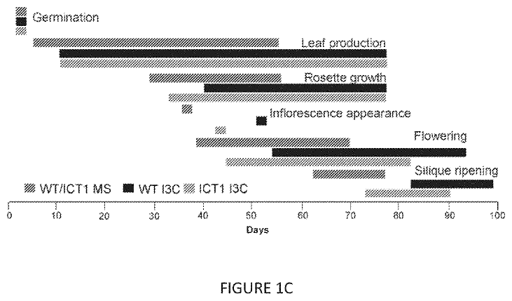

After three screening stages (see Materials and Methods hereinabove), one Indole-3-Carbinol Tolerant strain was identified, which was termed I3CT1. I3CT1 showed increased root elongation relative to WT on MS media supplemented with 400 μM I3C, while no difference in root length was observed when grown on MS (FIG. 1A, B).

In the absence of I3C, I3CT1 is phenotypically indistinguishable from WT. However, upon incubation with 400 μM I3C in the first two weeks of development, WT plants were twice as inhibited in the root-elongation assay than the I3CT1 plant. Upon subsequent transfer to I3C-free media, I3CT1 recovered faster than the WT: WT recovered to normal developmental rates two weeks following transfer, while I3CT1 resumed normal growth within one week (FIG. 1C).

### Example 2: Overexpression of S30 Gene Causes Tolerance to I3C

To identify the transgene carried by I3CT1, DNA was isolated and FOX-directed PCR was performed. I3CT1 was found to comprise the AT2G19750 gene which encodes a S30 ribosomal protein, a small (˜7 kDa) basic protein which is co-expressed with other ribosomal proteins (Carroll A J et al., 2008. Cellular Proteomics 7, 347-369). S30 is one of the 32 small 40S eukaryotic ribosomal subunit proteins, located in the “platform” region (Yusupova G and Yusupov M, 2017. ibid). In Arabidopsis, S30 is encoded by three paralogue genes located on different chromosomes; the three genes have about 76% identity between their cDNA sequences and encode identical proteins with differing expression frequency. The amino acid sequence of the S30 ribosomal protein, however, does not resemble the sequences of small ribosomal proteins other than the Arabidopsis paralogs. Of the three paralogs, AT2G19750 appears to have the highest expression (Barakat A et al., 2001, ibid). Overall, these genes are constitutively expressed at low levels in all vegetative tissues except pollen (Becker J D et al., 2003. Plant physiology 133, 713-725; Chen I.-P et al., 2003. The Plant Journal 35, 771-786).

To verify that tolerance to I3C is caused by the overexpression of AT2G19750 (S30) and not by the knockout of the underlying gene, additional lines predicted to overexpress AT2G19750 were analyzed. These new lines included two additional FOX transgenic lines, annotated as overexpressing S30 (obtained from RIKEN Japan, designated I3CT1 478, 8098), and independent transgenic lines generated in the course of the study as described in the Materials and Methods section hereinabove (designated I3CT1 1, 2, 28, 33). Wild Type Arabidopsis of a background of all transgenic plants (Col-0) and transgenic line with empty vector (NC(8)) served as controls.

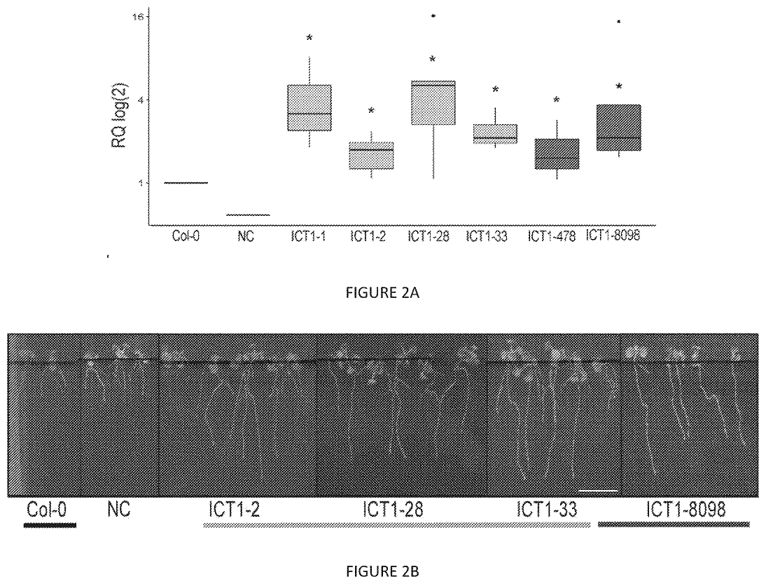

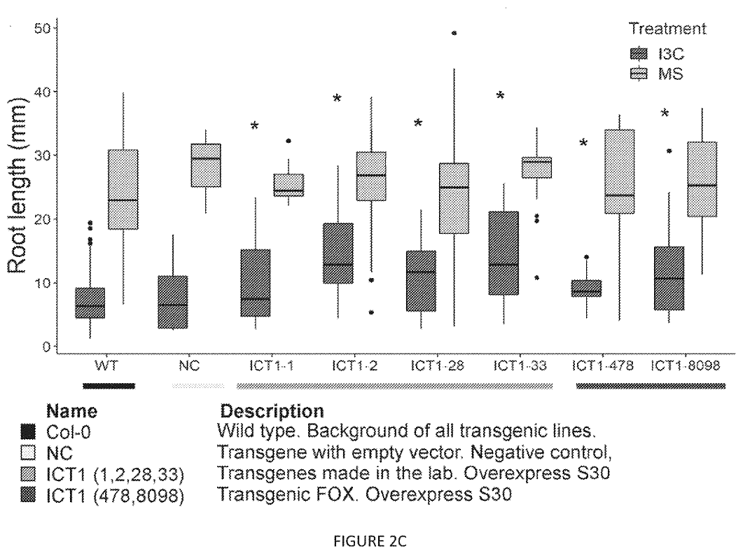

The various I3CT1 lines were analyzed for changes in AT2G19750 transcript levels. As seen in FIG. 2A, S30 transcript levels increase 1.7-6 fold in the two FOX lines I3CT⁸⁰⁹⁸ and I3CT⁴⁷⁸ and four transgenic strains, I3CT1², I3CT1²⁸, or I3CT1³³. All strains analyzed showed significantly increased root growth when grown on medium comprising 400 μM I3C (FIG. 2B, C), indicating that the tolerance is not a random result of fortuitous transgene integration, but rather is a result of the overexpression of the S30 gene. Strains I3CT1² and I3CT1⁸⁰⁹⁸ were selected for subsequent phenotypic analysis.

### Example 3: I3CT1 Plants are Tolerant to Exogenous I3C Up to 500 μM

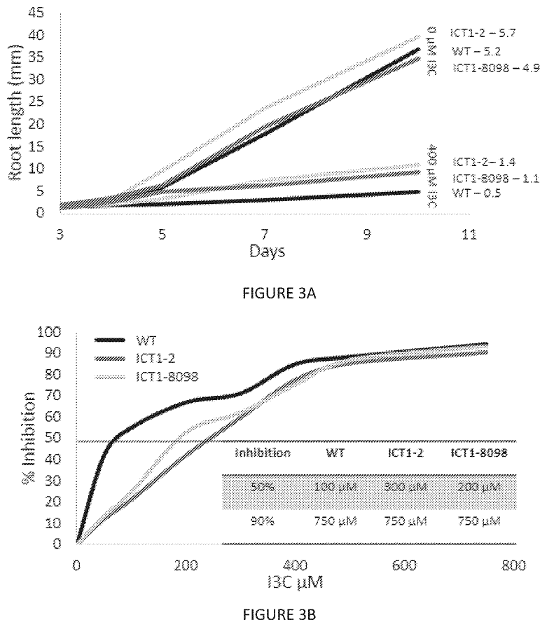

To calculate the degree of tolerance to I3C, growth rates of WT and the I3CT1 strains were determined for increasing concentrations of I3C. Root length was measured at several time points over increasing I3C concentrations (FIG. 3). The growth rate of the WT and the I3CT1 transgenic lines was similar on MS; however, a growth rate of 3 and 2.3 times faster was observed on 400 μM I3C for the transgenic lines I3CT1² and I3CT1⁸⁰⁹⁸ respectively. FIG. 3 demonstrates that all strains show maximum inhibition at 750 μM I3C. However, the growth rate of the WT was inhibited by 50% at about 50 μM while the growth rate of the I3CT1² and I3CR1⁸⁰⁹⁸ transgenic lines was inhibited by 50% only when the I3C concentration was 300 μM and 200 μM respectively. 90% inhibition was observed at 400 μM I3C for the WT but only at 750 μM and 500 μM for I3CT1² and I3CR1⁸⁰⁹⁸ respectively (FIG. 3).

### Example 4: I3CT1 Plants are Tolerant Specifically to I3C

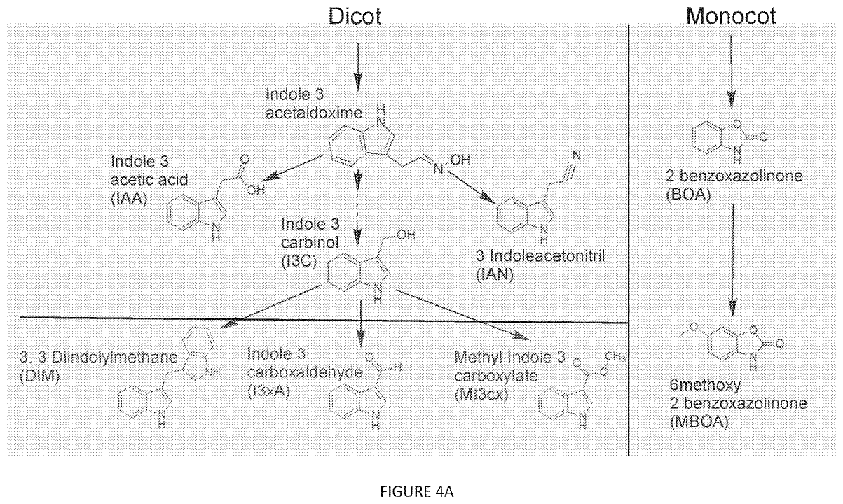

To test if overexpression of S30 causes tolerance specifically to I3C, or rather causes general tolerance to external chemical stress, root growth was tested with the addition of three groups of molecules to the growth medium: two subcategories of indole derivates: 1. Breakdown products of indol-3-acetaldoxime such as I3C, indol-3-acetic acid (IAA), and 3—Indoleacetonitril (IAN); and 2. breakdown products of I3C such as 3, 3Diindolylmethane (DIM), indole-3-carboxaldehyde (I3xA), and methyl Indole 3-carboxylate (MI3Cx). The third group contains benzoxazinoids: 2-Benzoxazolinon (BOA) and 6-Methoxy-2-benzoxazolinone (mBOA). Benzoxanoids are chemically unrelated to I3C, but with similar function of plant defense in gramineae (FIG. 4A). WT and the I3CT1 transgenic strains were assayed for root growth on 50% and 90% inhibition concentrations (IC) of the above molecules (Table 3).

TABLE 3
substance concentrations inhibiting
root elongation by 50% and 90%

Inhibition (μM)

| Substance | Name | 50% | 90% |
|---|---|---|---|
| Indole-3-acetic acid | IAA | 50 | 200 |
| Indole 3-acetonitril | IAN | 50 | 160 |
| Indole 3-carbinol | I3C | 100 | 400 |
| 3,3′-Diindolylmethane | DIM | 60 | 200 |
| Indole 3-Carboxalldehyde | I3xA | 140 | 300 |
| Methyl Indole 3-Carboxylate | MI3Cx | 200 | 375 |
| 2-Benzoxazolinon | BOA | 50 | 400 |
| 6-Methoxy-2-benzoxazolinone | mBOA | 50 | 400 |

As is seen in FIG. 4B, all chemicals assayed inhibited root growth in the WT.

I3CT1 did not exhibit any pronounced tolerance to IAN, I3CxA or MI3Cx, or the benzoxanoids. These results suggest that the tolerance of the I3CT1 line was limited to three chemicals: I3C, IAA and DIM, and is not a result of a general chemical-induced stress tolerance.

### Example 5: I3CT1 Plants are Transcriptionally Primed for Stress Response, Glucosinolate Synthesis and Transport

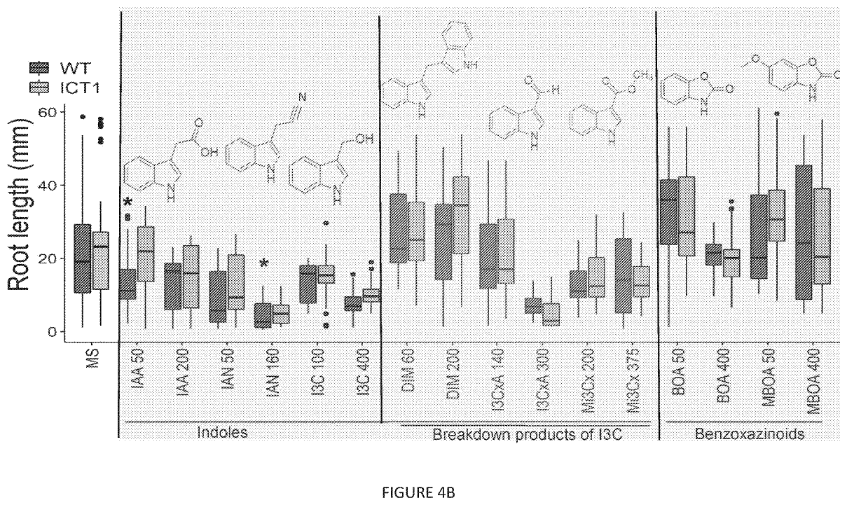

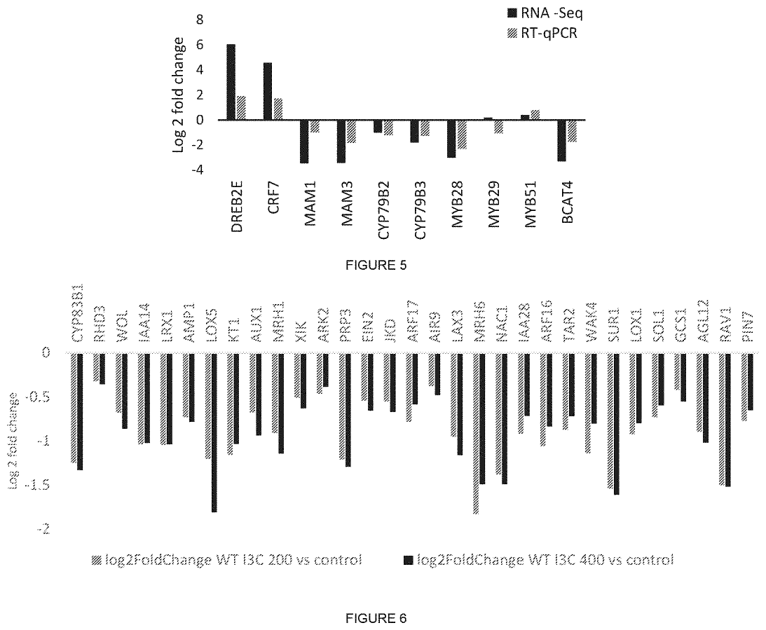

To garner understanding as to how treatment with I3C affects the plant, and how the overexpression of S30 influences the response to I3C, a transcript profiling approach was adopted to elucidate the effect of I3C on gene expression in A. thaliana. RNA was isolated in triplicates from 10-days-old Col-0, and the two independent transgenic lines, I3CT1² and I3CT1⁸⁰⁹⁸, each grown on MS or MS supplemented with 200 or 400 μM I3C, and analyzed by RNAseq using an Illumina HiSeq 2500 (using RNA from 24 samples, sequenced and analyzed). Following initial data analyses, several thousand genes were identified as I3C-responsive as reflected by the log 2 fold change values. Table 4 below shows the number of genes identified to be responsive to I3C and to have modified expression (up-or down-regulation) as a result of I3C application. To validate the I3C-induced changes in gene expression identified in Col-0, 10 transcripts were selected and subjected to RT-qPCR (FIG. 5).

TABLE 4
Numbers of genes identified as having modified expression

| Strain | | NT/200 | NT/400 | 200/400 |
|---|---|---|---|---|
| WT | Down | 2904 | 3697 | 31 |
| | Up | 3405 | 4134 | 84 |
| M2 | Down | 1857 | 3261 | 866 |
| | Up | 2700 | 3880 | 1504 |
| M8098 | Down | 1910 | ND | ND |
| | Up | 3222 | ND | ND |
| Treatment | | M2/WT | M8098/WT | M8098/M2 |
| NT | Down | 2181 | 1669 | ND |
| | Up | 2328 | 1537 | ND |
| 200 μM I3C | Down | 2575 | 2808 | 225 |
| | Up | 2421 | 2611 | 155 |
| 400 μM I3C | Down | 2758 | ND | ND |
| | Up | 2988 | ND | ND |

Table 4: The following comparisons were made: Top table: WT, I3CT² or I3CT⁸⁰⁹⁸ (the latter designated M2 and M8098, respectively), untreated compared to treated with 200 or 400 μM I3C. Bottom Table: The I3CT strains relative to WT or relative to each other under untreated conditions (NT), or treated with 200 or 400 μM I3C. ND=not done. “Down”, “Up” refer to type of the modified expression.

The transcriptome results were further analyzed in two steps. Functional enrichment analysis was conducted on different groups as follows: it was first asked which are the I3C-responsive genes in the WT. The majority of genes influenced are common to both I3C concentrations of 200 μM and 400 μM, with no significant change in expression intensities (data not shown). The gene ontology groupings identified in the common down-regulated genes included auxin-pathway genes, similar to that reported for an earlier study employing microarray technology (Katz E et al., 2015a. ibid), thus validating of the system used.

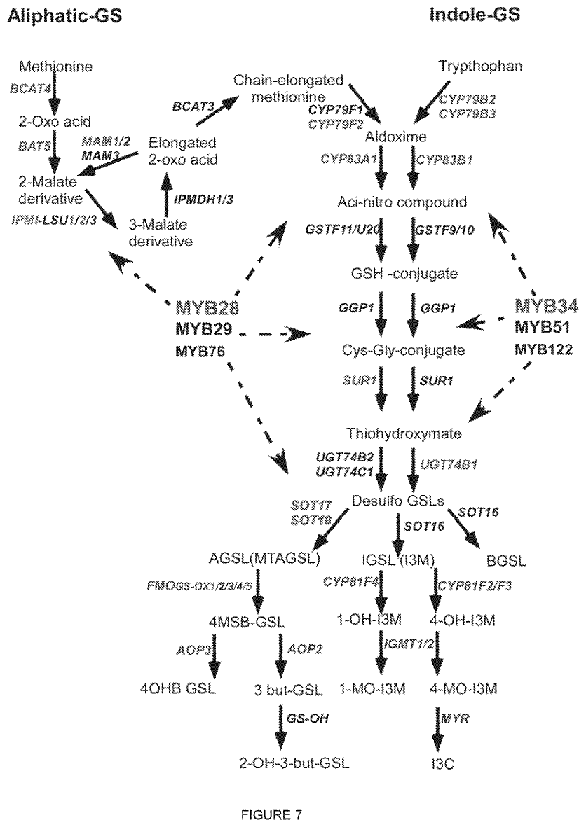

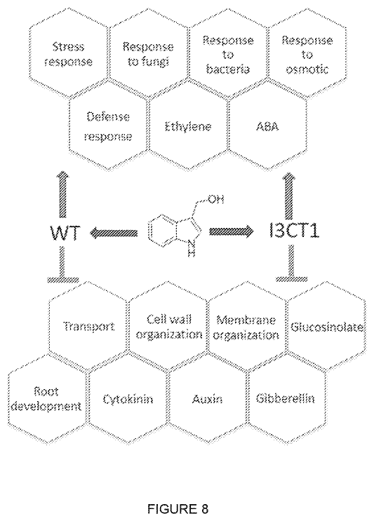

Additional ontology groupings influenced by both concentrations of I3C included root development (FIG. 6), glucosinolate metabolic process (FIG. 7), cytokinin, gibberellin, cell wall and transport-related genes. Up-regulated gene groups include genes known to be involved in the ethylene pathway and in responses to bacteria or fungi, proteasome activity, stress responses, cell death and apoptosis. ABA pathway and membrane-related genes also exhibited modified expression following I3C treatment (FIG. 8).

At 400 μM, there are 3 times more genes the expression of which has been regulated than at 200 μM. These additional genes did not define additional ontology families, rather additional genes in the ontologies identified at 200 μM. Thus, the additional stress of the higher concentration appears to have an additive effect on the number of genes with modified expression, rather than on influencing additional pathways (data not shown).

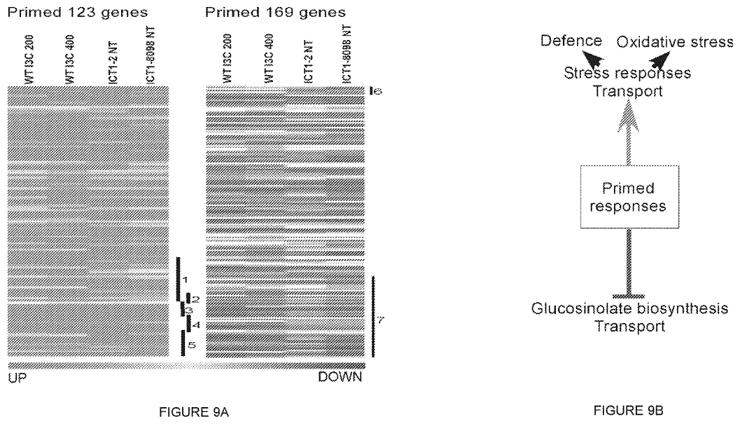

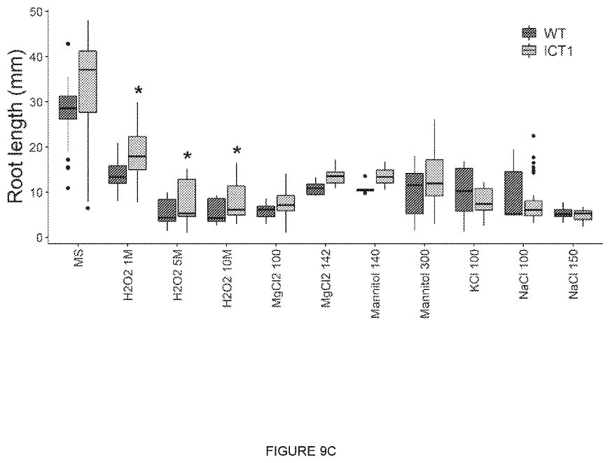

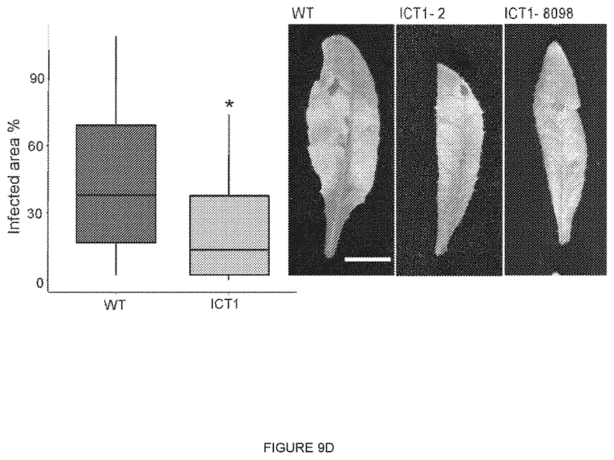

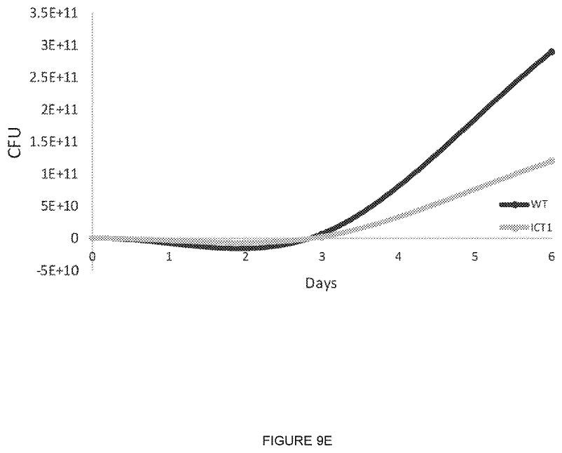

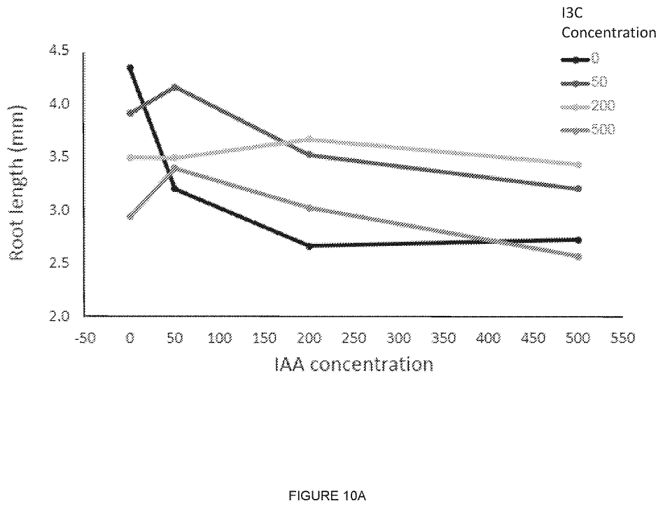

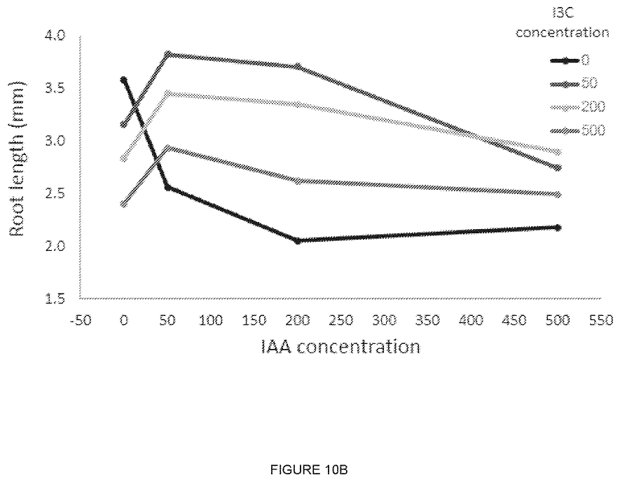

It was further examined whether the resistance to I3C in I3CT1 lines is manifested at the transcriptome level. In the absence of I3C, it was noticed that the two I3CT1 lines exhibited a modified regulation of a number of genes that are induced (123) or repressed (169) by I3C in the WT (FIG. 9A). These genes are referred to herein as “primed”. This primed group includes up-regulated genes involved in both abiotic (oxidative) and biotic (pathogen) stress responses. Glucosinolate biosynthesis and some transport functions are down-regulated in the I3C-tolerant strains (FIG. 9B). Most of the genes identified as I3C-responsive in WT also showed modified expression in I3CT1 lines following treatment with exogenous I3C. Moreover, a small number of genes are up (122) or down (45) regulated specifically in the I3CT1 lines but not in WT. Most of the up regulated genes are indicated in signaling defense and stress responses.

### Example 6: Resistance of I3CT1 Plants to Abiotic and Biotic Stress

To test whether the tolerance to I3C in I3CT1 plants is not a result of an inhibition of the I3C-induced transcriptional response, but rather due to a general transcriptional priming of stress responses, the growth-response of both I3CT1 strains to oxidative and pathogen stress was monitored. As seen in FIG. 9C, the root-length assay revealed that I3CT1 plants are tolerant to H₂O₂, though no enhanced tolerance to salt or osmotic stress was shown. The response to two pathogens, Botrytis cinerea and Pseudomonas PST DC3000 was monitored. As is demonstrated in FIG. 9D, in the WT plants, Botrytis infiltration lead to rapid and clearly visible necrosis of over 50% of the leaf area within 7 days. In two independent I3CT1 lines, a similar infiltration lead to partial necrosis, and in a relatively healthy leaf. To assay the response to Pseudomonas, colony forming units were counted over time from infected leaf discs. As seen in FIG. 9E, I3CT1 had 2.5 fold less colony forming units relative to WT. Thus, the priming of stress response genes identified above, manifests also in tolerance to both oxidative and biotic challenges.

### Example 7: I3C Acts as an Auxin Antagonist Both in Col-0 and in I3CT1

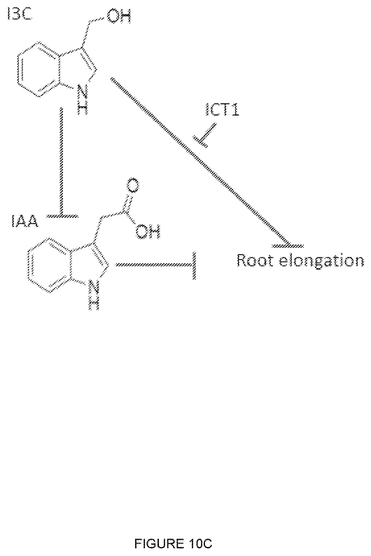

I3CT1 plants exhibited slight tolerance to IAA (FIG. 4). It was previously shown that I3C acts as an auxin antagonist on TIR1 (Katz et al., 2015b, ibid). The I3CT1 plants (transgenic lines I3CT1² and I3CT1⁸⁰⁹⁸ were assayed for response to exogenous auxin. Growth of WT on exogenous IAA cause concentration-dependent inhibition of root elongation (Katz et al., 2015b, ibid; Rahman A et al., 2007. The Plant Journal 50, 514-528). Addition of 50 and 200 μM I3C to IAA rescues the IAA phenotype, showing longer roots (FIG. 10A). Higher concentration of 500 μM I3C causes 95% inhibition by itself, therefor the rescue effect could not be observed. I3CT1 plants showed similar phenomenon, implying that I3C acts as an auxin antagonist both in Col-0 and in I3CT1 plants, such that the tolerance identified likely arises from a TIR1-independent mechanism.

### Example 8: Producing of Tobacco Plants Having Enhanced Tolerance to Plant Pathogens

### A. Growing Tobacco In Vitro

1. Sterilize seeds with a 10% commercial hypochlorite solution followed by 3 rinses in sterile DDW. Grow the seeds on ½ MS+0.8% agar individually in tubes.

2. When plants have developed a few leaves, use these leaves for transformation.

### B. The Transformation

1. Starter—Agrobacterium with the relevant plasmid
50 ml LB+50 μl Kanamycin+Agro
Grow for 2 days at 28° C. OD should be around 1

2. On the day of transformation, add 5 μl acetosyringone to the bacteria 4 hours before transformation and keep in shaker.

3. Prepare tobacco leaves: detach from the plant and make cuts. Keep the leaves in sterile water in a Petri dish or in a regeneration plate.

4. Centrifuge bacteria: 1400RCF for 20 minutes at 20° C.

5. Throw part of the supernatant and keep about 20 ml. Mix the bacteria with supernatant.

6. Put the bacteria in petri dish. Incubate the leaves for 5 min, rotate the dish for time to time

7. Blot the leaves on a sterile filter paper.

8. Transfer leaves to regeneration medium (MS+0.5 mg/L zeatin+0.05 mg/L IAA). Wrap with aluminum foil and keep in the growth room for 2 days.

9. Transfer the leaves to regeneration medium with selection and killing agent for Agrobacterium (MS+0.5 mg/L zeatin+0.05 mg/L IA; 50 mg/L kanamycin; 200 mg/L cefotaxime or 500 mg/l carbenicillin).

10. When regenerated tissues are big enough, transfer to MS in petri dishes then in tubes or magenta.

11. Rooting: water with sterile water.

12. Transfer to covered pots in growth chamber.

13. When vigor-enough plantlets are grown transfer to greenhouse.

Material and Media

Sterile 250 ml Erlenmeyer and 50 ml LB

### C. Selecting Tolerant Plants

The transgenic plants are infected with Botrytis cinerea or Pseudomonas as described hereinabove, and examined for enhanced tolerance phenotypically as described in Example 6.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-53. (canceled)

54. A plant or a part thereof comprising at least one cell modified to have enhanced expression and/or activity of an S30 protein compared to an unmodified cell, wherein the S30 protein comprises an amino acid sequence having at least 80% identity to the amino acid sequence set forth in SEQ ID NO:1, and wherein the plant has enhanced tolerance to at least one plant pathogen compared to a control plant not comprising modified cell or cells.

55. The plant or part thereof of claim 54, wherein the S30 protein is encoded by a polynucleotide comprising a nucleic acid sequence having at least 70% identity to the nucleic acid sequence set forth in SEQ ID NO:2.

56. The plant or part thereof of claim 54, wherein at least one exists:
a. the at least one modified cell having enhanced expression and/or activity of the S30 protein comprises an exogenous polynucleotide encoding said S30 protein, wherein the exogenous polynucleotide is selected from the group consisting of a polynucleotide endogenous to the plant cell and a polynucleotide heterologous to said plant cell; or
b. the at least one cell having enhanced expression and/or activity of the S30 protein comprises an up-regulated endogenous polynucleotide encoding said S30 protein.

57. The plant or part thereof of claim 54, wherein the expression and/or activity of the S30 protein or a polynucleotide encoding same within the at least one modified cell is enhanced by at least 50% compared to the expression and/or activity within the unmodified cell or cells.

58. The plant or part thereof of claim 54, wherein the at least one pathogen is selected from a plant pathogenic virus, fungus, bacterium, insect, oomycete, and nematode.

59. The plant of claim 54, wherein said plant is a crop plant.

60. A seed of the plant of claim 54, wherein a plant grown from the seed comprises at least one modified cell having enhanced expression and/or activity of an S30 protein compared to an unmodified cell, wherein the S30 protein comprises an amino acid sequence having at least 80% identity to the amino acid sequence set forth in SEQ ID NO:1, and wherein the plant has enhanced tolerance to at least one plant pathogen compared to a control plant not comprising modified cell or cells.

61. A tissue culture comprising at least one modified cell of the plant of claim 54 or a protoplast derived therefrom, wherein a plant regenerated from the tissue culture comprises at least one modified cell having enhanced expression and/or activity of an S30 protein compared to an unmodified cell, wherein the S30 protein comprises an amino acid sequence having at least 80% identity to the amino acid sequence set forth in SEQ ID NO:1, and wherein the plant has enhanced tolerance to at least one plant pathogen compared to a control plant not comprising modified cell or cells.

62. A method for conferring and/or enhancing tolerance of a plant or a part thereof to at least one plant pathogen, the method comprising enhancing the expression and/or activity of an S30 protein within at least one cell of the plant or part thereof, wherein the S30 protein comprises an amino acid sequence having at least 80% identity to the amino acid sequence set forth in SEQ ID NO:1, thereby conferring and/or enhancing tolerance of said plant or part thereof to the at least one pathogen compared to a control plant.

63. The method of claim 62, wherein the S30 protein is encoded by a polynucleotide comprising a nucleic acid sequence having at least 70% identity to the nucleic acid sequence set forth in SEQ ID NO:2.

64. The method of claim 62, wherein enhancing the expression and/or activity of the S30 protein comprises introducing into the at least one cell of the plant or part thereof an exogenous polynucleotide encoding said S30 protein, wherein the exogenous polynucleotide is selected from the group consisting of a polynucleotide endogenous to the at least one plant cell and a polynucleotide heterologous to said at least one plant cell.

65. The method of claim 64, wherein said method is selected from the group consisting of transforming the at least one cell with said polynucleotide or a construct comprising same and subjecting the at least one cell to genome editing using artificially engineered nucleases.

66. The method of claim 62, wherein enhancing the expression or activity of the 30S protein comprises up-regulating within the at least one cell of the plant or part thereof an endogenous polynucleotide encoding said S30 protein.

67. The method of claim 62, wherein the expression and/or activity of the S30 protein or a polynucleotide encoding same within the at least one modified cell is enhanced by at least 50% compared to the expression and/or activity within an unmodified cell.

68. The method of claim 62, wherein said method comprises conferring and/or enhancing tolerance to a plurality of plants to at least one plant pathogen.

69. The method of claim 68, wherein said method further comprises selecting plants showing an enhanced tolerance to the at least one plant pathogen compared to a control plant or to a pre-determined tolerance score value, wherein the control plant is a corresponding plant not manipulated to have an enhanced expression and/or activity of the S30 protein within at least one of the plant cells.

70. The method of claim 62, wherein the at least one plant pathogen is selected from a plant pathogenic virus, fungus, bacterium, insect, oomycete, and nematode.

71. The method of claim 62, wherein the plant is a crop plant.

72. An agricultural composition comprising an isolated protein comprising an amino acid sequence having at least 80% identity to SEQ ID NO:1, wherein the isolated protein is effective in enhancing the tolerance of a plant to at least one plant pathogen, further comprising at least one agriculturally acceptable diluent or carrier.

73. A method for enhancing and/or conferring tolerance of a plant or a part thereof to at least one plant pathogen, comprising contacting the plant or part thereof with the agricultural composition of claim 72.
